(19) United States
(12) Patent Application Publication
(10) Pub. No.: US 2024/0302276 A1
(43) Pub. Date: Sep. 12, 2024

(54) POLYPEPTIDE SEQUENCING AND FINGERPRINTING

(71) Applicant: Quantapore, Inc., South San Francisco, CA (US)

(72) Inventors: Milya DAVLIEVA, Alameda, CA (US); Martin HUBER, Menlo Park, CA (US)

(73) Assignee: Quantapore, Inc., South San Francisco, CA (US)

(21) Appl. No.: 18/569,128

(22) PCT Filed: Jun. 2, 2022
(86) PCT No.: PCT/US2022/072724

§ 371 Date: Dec. 11, 2023

Related U.S. Application Data
(60) Provisional application No. 63/202,401, filed on Jun. 9, 2021.

(51) Int. Cl. G01N 21/64; C12Q 1/37; G01N 33/487; G01N 33/68

(52) U.S. Cl. CPC G01N 21/6408; C12Q 1/37; G01N 21/6428; G01N 33/48721; G01N 33/6818; G01N 2021/6419; G01N 2021/6421; G01N 2021/6441

(57) ABSTRACT

In some aspects, the invention comprises a method for determining an amino acid sequence or fingerprint of a polypeptide. In some embodiments, the method comprises providing a solid state substrate comprising a cis side and a trans side, the substrate comprising a reaction well that defines a reaction volume and comprises (i) a proximal throughhole extending between the cis side and the trans side of the substrate, (ii) one or more side walls, and (iii) a distal opening. The solid state substrate further comprises an opaque metal layer that substantially blocks excitation light from penetrating into the reaction volume and from penetrating to the cis side of the substrate. Also provided is a carrier particle comprising a fluorescently labeled polypeptide strand that is attached to the carrier particle. The fluorescently labeled polypeptide strand comprises (i) a proximal end that is attached to the carrier particle, (ii) a distal end that is cleavable by an exopeptidase, and (iii) at least one fluorescently labeled amino acid comprising a fluorescent label. The carrier particle is located on the cis side of the substrate, but does not pass through the throughhole, such that the attached fluorescently labeled polypeptide strand protrudes through the throughhole so that the distal end of the fluorescently labeled strand is in the reaction volume. The trans side of the substrate is illuminated with excitation light to create a fluorescence excitation zone adjacent to the distal opening of the reaction well. While the substrate is illuminated, the fluorescently labeled polypeptide strand is reacted with an exopeptidase so that amino acids are released serially from the distal end of the stand and diffuse through the fluorescence excitation zone, so that fluorescently labeled amino acids in the excitation zone emit fluorescent signals. The fluorescent signals are detected as a function of time, whereby an amino acid sequence is determined from the time order of fluorescent signals detected from the released fluorescently labeled amino acids.

### CROSS REFERENCE

This application claims the benefit of U.S. Provisional Patent Application No. 63/202,401 filed Jun. 9, 2021, which is incorporated by reference herein in its entirety.

### INTRODUCTION

Proteomics is the study of the full complement of proteins from a given source, which may comprise an organism, a healthy or diseased tissue, or even a single cell. Since proteins are the primary effector molecules of living organisms, understanding their functions individually, in networks and at the proteome level is crucial for understanding states of health and disease. It is notable that most drugs act by increasing or decreasing the activity level of one or more target proteins and that proteome signatures (e.g. 2D-eletropherograms) are visibly altered in response to drug treatment, e.g. Anderson et al, Electrophoresis, 17: 443-453 (1996). Key tools for analyzing proteomes include 2D gel electrophoresis, chromatography, N-terminal Edman degradation, mass spectrometry and combinations of these techniques, Pappireddi et al, ChemBioChem 10.1002/cbic.201800650; Gevaert et al, Electrophoresis, 21: 1145-1154 (2000); Alfaro et al, Nature Methods, 18: 604-617 (2021). Despite the availability of these tools, strong interest has arisen in the application of single molecule nanopore-based methods to the detection and quantification of proteins and proteomes. In part this is because of the tremendous dynamic range of protein concentrations in typical samples (from picograms/milliliter to milligrams/milliliter), the relative lack of sensitivity of current methods, e.g. Restrepo-Perez et al, Nature Nanotechnology, 13:786-796 (2018); Luo et al, Int. J. Molec. Sci., 21: 2808 (2020); Kolmogorov et al, PLOS Computational Biology, 13(5): e1005356 (2017). Nanopore-based approaches have employed both electrical detection, e.g. Chinappi et al, J. Phys. Condens. Matter, 30: 204002 (2018); Han et al, Appl. Phys. Lett., 88: 093901 (2006); and optical detection, e.g. Ginkel et al, Proc. Natl. Acad. Sci., 115(13): 3338-3343 (2018), the latter of which relies on a FRET detection scheme and a complex molecular motor (with a FRET donor) to unfold and degrade labeled target polypeptides having the required C-terminal peptide tag.

In view of the limitations of electrical detection and the complexity of current optically based methods, the field of proteome analysis would be advanced by the availability of a simpler optically based single molecule detection method for identifying proteins and characterizing proteomes with important biomedical impact. Additionally, this approach may offer more than a million-fold improvement in sensitivity over conventional technologies.

### SUMMARY OF THE INVENTION

In some embodiments, the present disclosure provides a method for determining a amino acid sequence of a polypeptide. In some embodiments, the method comprises providing a solid state substrate comprising a cis side and a trans side, the substrate comprising a reaction well that defines a reaction volume and comprises (i) a proximal throughhole extending between the cis side and the trans side of the substrate, (ii) one or more side walls, and (iii) a distal opening. The solid state substrate further comprises an opaque metal layer that substantially blocks excitation light from penetrating into the reaction volume and from penetrating to the cis side of the substrate. Also provided is a carrier particle comprising a fluorescently labeled polypeptide strand that is attached to the carrier particle. The fluorescently labeled polypeptide strand comprises (i) a proximal end that is attached to the carrier particle, (ii) a distal end that is cleavable by an exopeptidase, and (iii) at least one fluorescently labeled amino acid comprising a fluorescent label. The carrier particle is located on the cis side of the substrate, but does not pass through the throughhole, such that the attached fluorescently labeled polypeptide strand protrudes through the throughhole so that the distal end of the fluorescently labeled strand is in the reaction volume. The trans side of the substrate is illuminated with excitation light to create a fluorescence excitation zone adjacent to the distal opening of the reaction well. While the substrate is illuminated, the fluorescently labeled polypeptide strand is reacted with an exopeptidase so that single amino acids are released serially from the distal end of the strand and diffuse through the fluorescence excitation zone, so that fluorescently labeled amino acids in the excitation zone emit fluorescent signals. The fluorescent signals are detected as a function of time, whereby an amino acid sequence is determined from the time order of fluorescent signals detected from the released fluorescently labeled amino acids.

In some embodiments, target polypeptides are treated with one or more denaturants and/or unfolding agents, such as surfactants, like sodium dodecyl sulfate (SDS) and reducing agents, like dithiothreitol (DTT), 2-mercaptoethanol (BME), for analysis by the method of the invention. SDS, an anionic compound that, in combination with heat and reducing agents, works to impart a nearly uniform negative charge density along the polypeptide. The resulting negatively charged peptide chain would translocate like a uniformly charged DNA molecule

In some embodiments, the distal opening of the reaction well has a minimum diameter of at least 30 nm. In some embodiments, the distal opening of the reaction well has a minimum diameter of 50 nm to 150 nm.

In some embodiments, the one or more walls of the reaction well are not tapered. In some embodiments, the one or more walls of the reaction well are substantially cylindrical.

In some embodiments, the opaque metal layer comprises gold or aluminum. In some embodiments, the opaque metal layer has a thickness of 100 nm to 600 nm. In some embodiments, the solid state substrate comprises a plurality of opaque metal layers.

In some embodiments, the reaction well has a well depth of at least 200 nm. In some embodiments, the reaction well has a well depth of 200 nm to 1000 nm.

In some embodiments, the fluorescently labeled polypeptide strand in the reaction volume comprises a fluorescently labeled polypeptide segment containing at least 60 contiguous amino acids, or in other embodiments, at least 100 contiguous amino acids.

In some embodiments, the throughhole has a minimum diameter of at least 2 nm. In some embodiments, the throughhole has a minimum diameter of 2 nm to 50 nm. In some embodiments, the substrate comprises a thin membrane layer that contains the proximal throughhole and that has a thickness of between 20 nm and 50 nm. In some embodiments, the thin membrane layer comprises silicon nitride.

In some embodiments, the excitation light has a wavelength of 380 nm or greater.

In some embodiments, the solid substrate comprises surface portion(s) that define the reaction volume, and the surface portion(s) comprise at least one surface passivation coating.

In some embodiments, one or more side walls of the reaction well comprise one or both of a silicon oxide coating and aluminum oxide coating.

In some embodiments, the fluorescently labeled polypeptide strand comprises at least two different kinds of amino acids, each kind labeled with a distinguishing fluorescent label.

In some embodiments, the carrier particle is not magnetic. In some embodiments, the carrier particle is magnetic.

In some embodiments, during said reacting, the carrier particle is maintained next to the proximal throughhole by a voltage bias.

In some embodiments, the carrier particle comprises a plurality of fluorescently labeled polypeptide strands having polypeptide sequences that are different from each other.

In some embodiments, after said reacting, the voltage bias is stopped to allow the carrier particle to move away from the proximal throughhole, so that the remaining fluorescently labeled polypeptide strand is removed from the reaction volume, and then a voltage bias is applied to move the same or a different carrier particle toward the proximal throughhole so that a new fluorescently labeled polypeptide strand is delivered into the reaction well for reacting with an exopeptidase.

In some embodiments, the solid state substrate comprises a plurality of reaction wells. In some embodiments, the plurality of reaction wells are configured as a one-dimensional or two-dimensional array. In some embodiments, two or more of the plurality of reaction wells each contain a fluorescently labeled polypeptide strand to be sequenced.

The above methods are also useful for determining amino acid sequences of a plurality of polypeptides.

In some embodiments, polypeptide analysis methods of invention comprise the steps: (a) translocating through a nanopore a labeled polypeptide wherein at least two different kinds of amino acids are labeled with different fluorescent labels (which are distinguishable), and wherein the nanopore comprises a passage through an insulative layer and an opaque layer, the passage through the opaque layer having a diameter through the opaque layer; (b) illuminating the passage from the direction of the opaque layer with a light beam having a wavelength greater than the diameter of the passage through the opaque layer, so that an excitation zone of non-propagating light is created within the passage through the opaque layer; (c) digesting the labeled polypeptide in the passage outside of the excitation zone to release amino acids one at a time at a rate less than the expected time of diffusion of the released amino acids out of the passage; (d) measuring a time series of fluorescent signals comprising signals from released labeled amino acids by detecting the signal generated by fluorescent labels as the released labeled amino acids diffuse out of the passage through the excitation zone. In some embodiments, a plurality of labeled polypeptides in a mixture is translocated through an array of a plurality of nanopores. In some embodiments, the plurality of nanopores is different than the plurality of labeled polypeptides. In some embodiments, the step of translocating further includes attaching labeled polypeptides to carrier particles wherein each carrier particle has a diameter greater that the diameter of the passage through the insulative layer so that carrier particles are prevented from entering the passage.

The present invention also provides kits for use in methods of the invention.

### BRIEF DESCRIPTION OF THE DRAWINGS

FIGS. 1A-1D illustrate features and operation of an exemplary reaction well of a solid state substrate of the invention.

FIG. 2 illustrates an exemplary sequencing apparatus of the invention.

### DETAILED DESCRIPTION OF EXEMPLARY EMBODIMENTS

While the invention is amenable to various modifications and alternative forms, specifics thereof are shown by way of example in the drawings and are described in further detail herein. It should be understood, however, that the intention is not to limit the invention to the particular embodiments described herein. On the contrary, the intention is to cover all modifications, equivalents, and alternatives falling within the spirit and scope of the invention.

For example, particular reaction well structures, particular labels, and fabrication examples are shown for purposes of illustration. It should be appreciated, however, that the disclosure is not intended to be limiting in this respect, as other structures, arrays of reaction wells, and other fabrication technologies that are not specifically detailed herein may be utilized to implement various aspects of the present inventions. Guidance for aspects of the invention is found in many references and treatises known to those with ordinary skill in the art, including, for example, Cao, Nanostructures & Nanomaterials (Imperial College Press, 2004); Levinson, Principles of Lithography, Second Edition (SPIE Press, 2005); Doering and Nishi, Editors, Handbook of Semiconductor Manufacturing Technology, Second Edition (CRC Press, 2007); Sawyer et al, Electrochemistry for Chemists, 2^{nd} edition (Wiley Interscience, 1995); Bard and Faulkner, Electrochemical Methods: Fundamentals and Applications, 2^{nd} edition (Wiley, 2000); Lakowicz, Principles of Fluorescence Spectroscopy, 3^{nd} edition (Springer, 2006); Hermanson, Bioconjugate Techniques, Second Edition (Academic Press, 2008); and the like, which relevant parts are hereby incorporated by reference.

In some aspects, the invention is directed to fluorescence-based analysis of polypeptides using sequential digestion of fluorescently labeled polypeptide by an exopeptidase activity.

Polypeptides may be obtained from any suitable sample source for sequencing using the present inventions. A wide variety of biological sources may be suitable, such as viruses, bacteria, mycobacteria, fungi, plants, animals, mammals, humans, etc. For complex organisms, such as humans, a variety of different sample types may be of interest, such as whole blood, plasma, serum, cells (such as red blood cells, white bloods cells, osteoclasts, osteoblasts, hepatocytes), urine, nasal mucus, feces, sputum, saliva, cerebral spinal fluid, mitochondria, exosomes, formalin fixed tissue samples, and tissue swabs.

Polypeptides may be prepared and/or purified using any appropriate method. Many methods are known for different sample types. For example, many reagents and kits are commercially available for isolating proteins from various sample types, such as Qiagen, Pierce, ThermoFisher, Bio-Rad, etc. The composition of protein extraction buffers can be altered to suit the properties of target proteins (e.g., solubility, hydrophobicity or hydrophilicity, pI, and the degree associated with membranes). Additionally, protein extraction can be targeted to specific organelles. Polypeptides may be isolated in their native state, or may be fragmented or modified in other ways prior to sequencing. For example, polypeptides may be fragmented by mechanical methods (e.g., by sonication or nebulization) or by enzymatic digestion.

Preferably, polypeptides are purified to substantially remove non-target polypeptides and other materials, such as cellular debris, nucleic acids, etc. In some embodiments, samples may be treated to remove some proteins before analyzing in accordance with methods of the invention. For example, albumin may be removed (or concentration reduced) from a blood sample prior to analyzing proteins therein. Methods for purifying polypeptides from various sample types are well known and are described, for example, in Twyman, Principle of Proteomics, 2^{nd} Ed. (Garland Science, 2014), and the like.

Fluorescently labeled polypeptide strands (also referred to as “fluorescently labeled strands”) for sequencing in accordance with the present inventions may be prepared by any suitable method. Each fluorescently labeled strand may have a proximal end and a distal end. The proximal end is coupled, directly or indirectly, to a carrier particle as described further below. The distal end of the fluorescently labeled strand protrudes away from the carrier particle when the fluorescently labeled strand is coupled to a carrier particle. Each fluorescently labeled strand is capable of being cleaved by an exopeptidase, so that single amino acids, some or all of which comprise fluorescent labels, are released serially (one-by-one) from the distal end of the strand for subsequent detection (discussed further below). In some embodiments, attachment of polypeptides to carrier particles and/or manipulation of polypeptides attached to carrier particles takes place in the presence of a protein denaturant or unfolding agent, such as SDS. Other exemplary unfolding agents for use in the method of the invention include, but are not limited to, Guanidine hydrochloride (GuHCL), CH₆CLN₃), and Urea (CO(NH₂)2. Most proteins are denatured in 8M Urea and most of them become randomly coiled in 6M guanidine chloride solution.

Target polypeptides are readied for translocation and cleavage by treatment with denaturants, including heat, surfactants and unfolding agents. Surfactants inhibit intra-strand hydrophobic interactions and reducing agents break intra-chain disulfide bonds. As a result, target polypeptides are linearized and provided with an approximately constant charge/mass ratio over their lengths. In some embodiments, surfactants are charged surfactants that have a hydrophilic-lipophilic balance value greater than 20 and a molecular weight in the range of between 200 and 500. Exemplary reducing agents for use in the method of the invention include, but are not limited to, dithiothreitol (DTT), dithioerythritol (DTE), L-glutathione (GSH), Tris (2-Carboxyethyl) phosphine hydrochloride (TCEP) and 2-mercaptoethanol.

In some embodiments, labeled polypeptides may be prepared for analysis by the same sample preparation protocols as used in SDS-polyacrylamide gel electrophoresis (or SDS-PAGE). An exemplary protocol for polypeptide preparation is as follows: Heat to 70° C. or 95° C. for 5 or 10 min in 2% (wt/vol; about 3.5 mM) SDS, with 0.2 M DTT as a reducing agent, so that proteins are completely denatured and all disulfide bonds are reduced. Each polypeptide binds, on the average, one SDS molecule per two amino acids (1.4 g of SDS per gram of protein). An alternative denaturation buffer may have the following ingredients: Heat to 70° C. or 95° C. for 5 or 10 min in SDS in a concentration in the range of 0.1-15 mM in Tris-Gly buffer (25 mM Tris/192 mM glycine, pH 8.4) at 15° C. In another embodiment, such denaturation buffer may comprise: 2 mg/ml protein with 1% SDS, 10% glycerol,

10 mM Tris-Cl, pH 6.8, 1 mM ethylene diamine tetraacetic acid (EDTA), dithiothreitol (DTT) or 2-mercaptoethanol, optionally with 1 mM ethylene diamine tetraacetic acid (EDTA). In some cases, protein could be first treated with TCEP (5 mM) at room temperature for 30 min to break disulfide bonds and subsequently denatured at 90° C. for 5 min in PBS (phosphate-buffered saline) with 2% SDS. Alternatively, protein (0.5-12 mg/ml) could be dissolved initially in 6 M Guanidinium Hydrochloride (GuHCL) and 0.1% BME to form the polypeptide chain in a random coil configuration. Further guidance for denaturant buffers may be found in Wiesner et al, Electrophoresis, 42: 206-218 (2021), and Gudiksen et al, Proc. Natl. Acad. Sci., 103(21): 7968-7972 (2006).

In some embodiments, a fluorescently labeled polypeptide strand comprises one kind of amino acid that is labeled with a fluorescent label. In some embodiments, a fluorescently labeled polypeptide strand comprises two different kinds of amino acids, each kind labeled with a distinguishing fluorescent label. In some embodiments, a fluorescently labeled polypeptide strand comprises at least two different kinds of amino acids, each kind labeled with a distinguishing fluorescent label. For example, Cysteines (Cys) and Lysines (Lys) may be labeled with fluorescent dyes. In such embodiments, a collection of fluorescent signals from the polypeptide strand may form Cys/Lys fingerprint that may provide enough information to identify the most proteins in the human proteome database (Y. Yao, Phys. Biol, 2015). In some embodiments, a fluorescently labeled polypeptide strand comprises three different kinds of amino acids, each kind labeled with a distinguishing fluorescent label. In some embodiments, a fluorescently labeled polypeptide strand comprises at least three different kinds of amino acids, each kind labeled with a distinguishing fluorescent label. For example, amino acids lysine (Lys), cysteine (Cys), and methionine (Met) may be labeled with three spectrally-resolvable fluorophores using different chemistry. The primary amines in Lys may be labeled with NHS esters, thiols in Cys with maleimide groups, and Met may be labeled using the redox-based approach (S. Lin et all, Science 2017). In such embodiments, a collection of 3-color optical fingerprints will provide sufficient information to accurately identify the entire human proteome with >95% accuracy (S. Ohayon PLOS Computational Biology, 2019). In some embodiments, a fluorescently labeled polypeptide strand comprises four different kinds of amino acids, each kind labeled with a distinguishing fluorescent label. In some embodiments, phosphoserines may be fluorescently labeled to allow single molecule sequencing of phosphorylated serine sites (J. Swaminathan Nat Biotechnology, 2019).

In some embodiments, a fluorescently labeled polypeptide strand comprises a single fluorescent label attached to a single kind of monomer, for example, every cysteine (or substantially every cysteine) of a polypeptide strand is labeled with a fluorescent label, e.g. a cyanine dye. In such embodiments, a collection, or sequence, of fluorescent signals from the polypeptide strand may form a signature or fingerprint for the particular polypeptide. In some such embodiments, such fingerprints may or may not provide enough information for a sequence of monomers to be determined or an identity of a polypeptide to be determined, for example, by reference to a protein database. In such embodiments, such fingerprints may comprise a series of time measurements between detection events of labeled amino acids passing through a detection zone.

Fluorescent labels include any fluorescent dyes chosen by the user for identifying attached amino acids in the methods of the invention. Exemplary fluorescent labels include, but are not limited to, xanthenes, fluoresceins, rhodamines, sulforhodamines, rhodals, cyanines, coumarins, and pyrenes. If different fluorescent labels are used to identify and distinguish different kinds of amino acids, then the fluorescent labels can be from the same structural class of fluorescent labels (e.g., all are fluoresceins) or from different classes of fluorescent labels.

In some embodiments, exemplary fluorescent labels may be selected from rhodamine dyes, fluorescein dyes and cyanine dyes. In some embodiments, fluorescent dyes may comprise two or more dyes selected from Oregon Green 488, Fluorescein-EX, fluorescein isothiocyanate, Rhodamine Red-X, Lissamine rhodamine B, Calcein, fluorescein, rhodamine, one or more BODIPY dyes, Texas Red, Oregon Green 514, and one or more Alexa Fluors. Exemplary BODIPY dyes include BODIPY FL, BODIPY R6G, BODIPY TMR, BODIPY 581/591, BODIPY TR, BODIPY 630/650 and BODIPY 650/665. Exemplary Alexa fluorescent labels include Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 635, Alexa Fluor 647, Alexa Fluor 660, Alexa Fluor 680, Alexa Fluor 700, Alexa Fluor 750 and Alexa Fluor 790.

In further embodiments, exemplary fluorescent labels include, but are not limited to, Alexa 488, AMCA, Atto 655, Cy3, Cy5, Evoblue 30, fluorescein, Gnothis blue 1, Gnothis blue 2, Gnothis blue 3, Dy630, Dy635, MR121, rhodamine, Rhodamine Green, Oregon Green, TAMRA, and the like.

In further embodiments, exemplary fluorescent labels for labeling amino acids, include, but are not limited to, Oregon Green 488, fluorescein-EX, FITC, Rhodamine Red-X, Lissamine rhodamine B, calcein, fluorescein, rhodamine, BODIPYs, and Texas Red, e.g. which are disclosed in Molecular Probes Handbook—A Guide to Fluorescent Probes and Labeling Technologies, 11th Edition (2010) as revised online as of the date of the present disclosure.

The carrier particles have dimensions that are sufficiently large to prevent the carrier particles from moving through the throughholes of the reaction wells into the reaction wells. Each carrier particle is capable of being moved by an electromagnetic force to be near a throughhole of a reaction well to deliver the distal end of a first fluorescently labeled polypeptide strand, which is attached to the carrier particle, through the throughhole into a reaction well. In some embodiments, the carrier particles are magnetic carrier particles. In some embodiments, the carrier particles are not magnetic carrier particles. In some embodiments, the electromagnetic force is a voltage bias. In some embodiments, the electromagnetic force is a magnetic force.

In some embodiments, carrier particles have a diameter, or a largest diameter, that is at least 15 nm, or at least 20 nm, or at least 25 nm, or at least 30 nm, or from 15 nm to 100 nm, or from 15 nm to 75 nm, or from 15 nm to 50 nm, or from 20 nm to 50 nm. However, carrier particles having larger or smaller diameters may also be used. As used herein, “nanoparticle” refers to a carrier particle having a diameter, or largest diameter, that is less than 200 nm, or less than 150 nm, or less than 100 nm.

The carrier particles may be charged or uncharged. The carrier particles may have a net neutral charge, a net positive charge, or a net negative charge, based on the net balance of positively charged groups and negatively charged groups on the particles under the pH conditions of the surrounding aqueous medium, which usually comprises an aqueous buffer. Preferably, for control of movement by an electric field (voltage bias), the carrier particles have a net negative charge when they comprise one or more attached fluorescently labeled polypeptide strands.

In some embodiments, each carrier particle is capable of being moved by an electromagnetic force away from a throughhole of a reaction well, to remove a cleaved fluorescently labeled polypeptide strand from the throughhole. In preferred embodiments of methods of the present invention, after exopeptidase cleavage of a fluorescently labeled polypeptide strand in a well, the carrier particle is moved away from the well either actively by reversing an electric field or passively via diffusion, and then the same or a different carrier particle is moved near the throughhole of the well to deliver the distal end of a second (sometimes called “new”) fluorescently labeled polypeptide strand through the throughhole into the reaction well. To facilitate movement of distal ends of fluorescently labeled polypeptide strand into and out of the reaction wells by voltage bias or magnetic field, the carrier particles are not covalently coupled to the throughholes.

In some embodiments, the carrier particles comprise spherical particles. In some embodiments, the carrier particles comprise non-spherical particles, having, for example, elliptoid or irregular shapes. In some embodiments, the carrier particles comprise both spherical particles and non-spherical particles. In some embodiments, the carrier particles are provided as a uniform population of substantially identical carrier particles within a size range (e.g., within plus or minus a standard deviation or coefficient of variation), but the carrier particles are not necessarily identical, provided that they effectively carry and deliver fluorescently labeled polypeptide strands to the reaction wells.

Carrier particles may be made from any materials that are suitable for the purposes of the present invention. In some embodiments, the carrier particles are metal particles, such as metal nanoparticles. In some embodiments, the carrier particles are gold nanoparticles. In some embodiments, the carrier particles are silver nanoparticles. In some embodiments, one or more carrier particles comprise one or more magnetic materials, such as iron or iron oxide, that allow the particles to be moved by a magnetic field. In some embodiments, the carrier particles are iron oxide particles. In some embodiments, the carrier particles are silica particles or controlled pore glass particles.

In some embodiments, the carrier particles comprise an immobilized protein, such as streptavidin or an antigen-specific antibody, for binding a biotin moiety or antigen moiety that is attached to or associated with a fluorescently labeled polypeptide strand to be sequenced. In some embodiments, the carrier particles are proteins, such as streptavidins or antibodies, for binding one or more biotinylated polypeptides or antigen-polypeptide conjugates.

Fluorescently labeled polypeptide strands may be attached to carrier particles by any suitable means. Usually, fluorescently labeled polypeptide strands are attached to carrier particles by means of a capture moiety. Capture moieties usually comprise members of a pair of moieties that have a mutual affinity for each other (also referred to as a “binding pair”). In some embodiments, capture moieties are polypeptides such as antibodies or antigens. In some embodiments, capture moieties are oligonucleotides.

Capture moieties may be monovalent (for capturing one binding partner) or multivalent (for capturing multiple binding partners). Avidin is an example of a monovalent capture moiety, and streptavidin, with four biotin binding sites, is an example of a multivalent capture moiety. In some embodiments, the capture moiety comprises an antibody (for specifically binding one or two antigens). More generally, the capture member may be any member of a binding pair for which the other member of the pair is associated with a fluorescently labeled polypeptide strand to facilitate attachment of the fluorescently labeled polypeptide strand to the carrier particle.

Usually, if a carrier particle is not itself a capture moiety (e.g., if the carrier particle is not streptavidin, an antibody, or another monovalent or multivalent entity), the carrier particle comprises at least one, and preferably a plurality, of capture moieties by which fluorescently labeled polypeptide strands may be attached directly or indirectly to the carrier particles.

Capture moieties are usually attached to carrier particles by linkers. Any suitable linker may be used. Since exopeptidase cleavage and other elements of the present invention are usually performed in aqueous solution, linkers are usually hydrophilic. Exemplary linkers include polymers such as polyethylene glycol, polyamides, poly(polyethylene glycol phosphates), polyalkyl phosphates, polyamines, and the like. Such linkers may have any suitable length. Some illustrative linkers and conjugation methods are described in Example 1 below.

Exemplary functional group pairs and their resulting linkages for attaching capture moieties to carrier particles are shown in Table 1 below.

TABLE 1

| Carrier | | |
|---|---|---|
| Particle | Polypeptide | Linkage |
| amino | NHS (N-hydroxy succinimide) | amide —NH—C(═O)- |
| amino | carboxyl | amide —NH—C(═O)- |
| carboxyl | amino | amide —C(═O)NH— |
| thiol | thiol | disulfide-S—S— |
| gold | thiol | gold thiolate Au—S— |
| azide | DBCO or BCN | cycloaddition adduct |
| maleimide | thiol | Michael adduct |

Fluorescently labeled polypeptide strands are immobilized on (attached to) carrier particles by any suitable means, such as the protein attachment chemistries disclosed by Deng et al, Nature Comm. Chem., (2020) 3:67 (https://doi.org/10.1038/s42004-020-0309-y); Onoda et al, ChemBioChem 10.1002/cbic.201900692 (2020); Theile et al, Nat Protoc., 8(9): 1800-1807 (2013); Rosen et al, Nature Chemical Biology, 13: 697-701 (2017); Camarero, Peptide Science, 90(3): 450-458 (2007); Camarero et al, U.S. Pat. No. 7,972,827; and the like; which references are incorporated herein by reference. Of special interest is the attachment of polypeptides to carrier particles by the N-terminus of the polypeptides as taught by the above references.

As noted above, fluorescently labeled polypeptide strands of the invention are cleaved using one or more exopeptidases. An exopeptidase is selected to have substantially no endopeptidase activity, to ensure that the exopeptidase cleaves only single, consecutive amino acids from the distal end of the fluorescently labeled strand.

Any suitable exopeptidase may be used. Exopeptidases may be native (i.e., have a chemical structure found in nature) or modified relative to their native structures, and may be from their natural sources or from recombinant hosts. For example, exopeptidase may be chemically modified after isolation or purification, and may also be generated by combinatorial and recombinant techniques, including for example screening for exopeptidases with desired properties. Similarly, it might be possible to engineer the enzyme by genetic substitutions of amino acid side-chains located at binding sites to achieve significant improvement in performance. An exemplary exopeptidase is carboxypeptidase Y (CPD-Y, ThermoFisher) or Maltcarboxypeptidase II (CPD-M-II). In some embodiments, two peptidases with different specificities can be employed with advantage. For example, since CPD-Y has a preference for hydrophobic residues and CPD-M_II for basic, both of them may be used to achieve the intended results. Alternatively, the data from different exopeptidases may be collected and merged. Exopeptidases other than those considered here may be used as well with similar success. For example, leucine aminopeptidases (LAP) that can cleave a range of amino acid types may be used to provide additional sequence information.

In some aspects of the inventions disclosed herein, a solid state substrate comprises a cis side and a trans side. The substrate comprises a reaction well that defines a reaction volume. The reaction well comprises (i) a proximal throughhole extending between the cis side and the trans side of the substrate, (ii) one or more side walls, and (iii) a distal opening. The solid state substrate further comprises an opaque metal layer that substantially blocks excitation light that is incident on the trans side of the substrate from penetrating into the reaction volume of the reaction well and from penetrating to the cis side of the substrate.

Reaction wells for containing fluorescently labeled polypeptide strands to be sequenced may have any of a variety of shapes and sizes. For example, although cylindrical wells with circular cross-sections and parallel side walls are suitable, reaction wells may also have elliptical, triangular, square, rectangular, pentagonal, hexagonal, octagonal or other regular or irregular cross-sectional shapes, with parallel or non-parallel side walls. For example, the side walls of reaction wells having any of the foregoing shapes may be parallel, tapered, truncated-conical, or hour-glass shaped. For example, a cylindrical well may be considered to have a single side wall that is inherently parallel with itself.

Reaction wells may have any of a variety of dimensions that may be chosen by the user. The choice of specific dimensions can take into consideration a selected length and a minimum diameter of the fluorescently labeled strands that will be sequenced, whether the fluorescently labeled strands are in single- or double-stranded form, and any other relevant considerations.

The depth and minimum diameters of reaction wells are usually selected so that each reaction well can contain (1) a distal end of a fluorescently labeled polypeptide strand to be sequenced and also (2) an exopeptidase molecule that is bound to the distal end of the strand during proteolytic cleavage of terminal amino acids.

As used herein, “minimum diameter” means the shortest diameter of a reaction well or of a throughhole, as applicable. For example, a cylinder has a single diameter, which is the minimum diameter. For a reaction well having a square overhead cross-section that is perpendicular to the depth axis of the reaction well, the minimum diameter is the distance between (and perpendicular to) two opposing walls of the reaction well (the length of a side of the square cross-section), whereas the maximum diameter is the length of a diagonal across the square cross-section. For a reaction well having tapered or other non-parallel walls, the minimum diameter of the reaction well is the shortest dimension in a cross-section of the well. More generally, the distal opening, and at least a portion of the reaction well extending from the distal opening, have a minimum diameter that satisfies requirements (1) and (2) above. Therefore, if the distal opening of a reaction well has a particular minimum diameter, then the minimum diameter of at least a portion of, or all of, the reaction well extending from the distal opening towards the throughhole of the well is equal to or greater than the minimum diameter of the distal opening of a reaction well.

In some embodiments, the reaction well, or the distal opening of the reaction well, has a minimum diameter of at least 30 nm, or at least 40 nm, or at least 50 nm, or at least 60 nm, or at least 70 nm, or at least 80 nm, or at least 90 nm, or at least 100 nm. In some embodiments, the reaction well has a minimum diameter that is less than 150 nm, or less than 120 nm, or less than 100 nm, or less than 90 nm, or less than 80 nm. In some embodiments, the reaction well has a minimum diameter of 30 nm to 250 nm, or 30 nm to 150 nm, or 30 nm to 120, or 30 nm to 100, or 30 nm to 90 nm, or 50 nm to 150 nm, or 50 nm to 120 nm, or 50 nm to 100 nm, or 50 nm to 90 nm, or 80 nm to 120 nm.

In some embodiments, the reaction well has a well depth of at least 150 nm, or at least 200 nm, or at least 300 nm, or at least 400 nm, or at least 500 nm. In some embodiments, the reaction well has a well depth that is less than 1000 nm, or is less than 800 nm, or is less than 700 nm, or is less than 600 nm, or is less than 500 nm. In some embodiments, the reaction well has a well depth of 150 nm to 1000 nm, or 150 nm to 800 nm, or 150 nm to 700 nm, or 150 nm to 600 nm, or 150 nm to 500 nm. In some embodiments, the reaction well has a well depth of 200 nm to 1000 nm, or 200 nm to 800 nm, or 200 nm to 700 nm, or 200 nm to 600 nm, or 200 nm to 500 nm. In some embodiments, the reaction well has a well depth of 300 nm to 1000 nm, or 300 nm to 800 nm, or 300 nm to 700 nm, or 300 nm to 600 nm, or 300 nm to 500 nm. In some embodiments, the reaction well has a well depth of 400 nm to 1000 nm, or 400 nm to 800 nm, or 400 nm to 700 nm, or 400 nm to 600 nm, or 400 nm to 500 nm.

As noted above, the reaction well also comprises a proximal throughhole extending between the cis side and the trans side of the substrate. Here, “proximal” throughhole means that the throughhole is closer to the cis side of the substrate than is the distal opening of the associated reaction well. Each proximal throughhole has a minimum diameter that is (1) sufficiently large to allow the distal end of a fluorescently labeled polypeptide strand to be drawn into and through the throughhole, and (2) is sufficiently small to prevent the carrier particle to which the fluorescently labeled strand is attached from passing through the throughhole to the trans side of the substrate. Preferably, the minimum diameter of the proximal throughhole is sufficiently small to prevent any exopeptidase molecules from passing through the proximal throughhole from the trans side to the cis side of the substrate. In some embodiments, each proximal throughhole has a minimum diameter that is smaller than the smallest dimension of the exopeptidase.

In some embodiments, the proximal throughhole has a minimum diameter of at least 2 nm. In some embodiments, the proximal throughhole has a minimum diameter of 2 nm to 50 nm, or 3 nm to 50 nm, or 5 nm to 50 nm, or 10 nm to 50 nm, or 20 nm to 50 nm, or 2 nm to 40 nm, or 3 nm to 40 nm, or 5 nm to 40 nm, or 10 nm to 40 nm, or 20 nm to 40 nm, or 3 nm to 30 nm, or 5 nm to 30 nm, or 10 nm to 30 nm, or 2 nm to 20 nm, or 3 nm to 20 nm, or 5 nm to 20 nm, or 10 nm to 20 nm, or 2 nm to 10 nm, or 3 nm to 10 nm, or 5 nm to 10 nm.

In some embodiments, the proximal throughhole has a longitudinal thickness of at least 10 nm, or at least 15 nm, or at least 20 nm, or from 10 to 60 nm, or from 10 to 50 nm or from 10 to 40 nm, or from 20 to 60 nm, or from 20 to 50 nm, or from 20 to 40 nm, or from 30 to 60 nm, or from 30 to 50 nm.

In some embodiments, the substrate comprises a thin membrane layer that contains the proximal throughhole. In some embodiments, the thin membrane layer comprises silicon nitride (SiN).

Substrates comprising reaction wells for use in the present inventions may be fabricated by any suitable method, in various forms of solid materials including but not being limited to silicones (e.g. Si₃N₄, SiO₂), metals, metal oxides (e.g. Al₂O₃) plastics, glass, semiconductor material, and combinations thereof. Fabrication techniques for making solid state substrates can be found in the following exemplary references that are incorporated by reference: Golovchenko et al, U.S. Pat. No. 6,464,842; Sauer et al, U.S. Pat. No. 7,001,792; Su et al, U.S. Pat. No. 7,744,816; Meller et al, International patent publication WO2009/020682; Yan et al, Nano Letters, 5(6): 1129-1134 (2005); Wanunu et al, Nano Letters, 7(6): 1580-1585 (2007); Dekker, Nature Nanotechnology, 2: 209-215 (2007); Storm et al, Nature Materials, 2: 537-540 (2003); Zhe et al, J. Micromech. Microeng., 17: 304-313 (2007); and the like.

The solid state substrate comprises one or more light-blocking layers referred to herein as opaque metal layers. Each opaque metal layer reflects and/or absorbs incident light from the excitation beam, thereby (1) protecting the fluorescently labeled strands in the reaction well and on the cis side of the substrate from photobleaching and from other damage caused by incident light, and (2) preventing incident light from causing labels in the fluorescently labeled strands to fluoresce before being cleaved from the labeled strand by the exopeptidase, potentially interfering with the correct fluorescent signals from cleaved fluorescently labeled amino acids.

An opaque metal layer may comprise Sn, Al, V, Ti, Ni, Mo, Ta, W, Au, Ag or Cu, for example, and/or alloys or combinations thereof. In some embodiments, an opaque metal layer comprises Al, Au, Ag or Cu. In some embodiments, an opaque metal layer comprises aluminum (Al) or gold (Au). The composition of the opaque metal layer may be selected based on the wavelength-dependence of the metal's reflectance of incident light. For fluorescence detection in the present invention, incident light is typically in the visible spectrum in the range of from about 380 nm to about 740 nm. Aluminum exhibits a reflectance of about 90% across the entire visible spectrum, making it a good candidate for use as an opaque layer. Gold exhibits a reflectance of about 35% for wavelengths between about 260 nm to about 480 nm and then rises sharply for wavelengths between about 480 nm to 700, exceeding about 90% for wavelengths greater than about 550 nm. Thus, gold has good light blocking characteristics across the visible spectrum, especially for wavelengths above about 480 nm, particularly in the red and infrared regions. Silver has a reflectance above about 80% for wavelengths above about 350 nm.

In some embodiments, the substrate comprises two or more opaque metal layers. For example, the substrate may comprise a gold layer and an aluminum layer, both of which reflect and/or absorb incident light impinging on the trans side of the substrate. In some embodiments, the substrate comprises a gold layer over an aluminum layer, such that the aluminum layer is closer to the cis side of the substrate than is the gold layer. One benefit of having a gold top layer (which may also be referred to as an outer gold layer or distal gold layer, and which surrounds the distal opening of the reaction well) is that gold can enhance the intensity of light excitation around the distal opening of the reaction well, thereby increasing the yield of fluorescent signals from each released fluorescently labeled amino acid that diffuses through the excitation zone. In some embodiments, the substrate comprises a distal aluminum layer over a gold layer, such that the gold layer is closer to the cis side of the substrate than is the aluminum layer.

The thickness of an opaque metal layer may vary and depends on the physical and chemical properties of material composing the opaque layer. In some embodiments, the thickness of an opaque layer may be at least 40 nm, or at least 80 nm, or at least 120 nm, or at least 200 nm, or at least 300 nm. In other embodiments, the thickness of an opaque layer may be in the range of from 50 to 700 nm; in other embodiments, the thickness of an opaque layer may be in the range of from 100 to 600 nm. If the substrate comprises more than one opaque metal layer, then “thickness” refers to the thickness of each individual layer.

An opaque metal layer need not block (i.e. reflect or absorb) 100 percent of the light from an excitation beam. In some embodiments, the opaque metal layer, or plurality of opaque metal layers if more than one layer is present, blocks at least 30%, at least 50%, or at least 90%, or at least 95%, or at least 99%, or at least 99.5%, or at least 99.9% of the excitation light that is incident on the distal opening of a reaction well at a depth that is 50 nm from the proximal throughhole of the reaction well.

Opaque layers may be fabricated by a variety of techniques. Material deposition techniques may be used including chemical vapor deposition, electrodeposition, epitaxy, thermal oxidation, physical vapor deposition, including evaporation and sputtering, casting, for example. In some embodiments, atomic layer deposition may be used, e.g. U.S. Pat. No. 6,464,842; Wei et al, Small, 6(13): 1406-1414 (2010), which are incorporated by reference.

The solid state substrate may comprise other layers. For example, a solid state substrate may comprise one or more non-opaque layers to increase the depth of the reaction well(s) in the substrate. Such a dielectric layer may comprise SiO₂, TiO₂, for example.

In some embodiments, a solid state substrate may comprise a thin adhesive layer between two other layers to enhance stability of the layers and deter delamination or other kinds of damage. For example, including a thin adhesive layer between a gold layer and an aluminum layer can enhance the adherence of the gold layer in the solid state substrate, as taught by Aouani et al., ACS Nano 3(7):2043-2048 (2009). Such a thin adhesive layer may comprise any suitable material, including for example chromium, titanium, titanium dioxide, chromium oxide, or nickel. A thin adhesive layer may have any suitable thickness. For example, a thin adhesive layer may have a thickness from 1 nm to 40 nm, or 5 nm to 20 nm. In some embodiments, when a thin adhesive layer is tens or hundreds of nanometers from the distal opening of the reaction well, the thin adhesive layer has negligible or no effect on the fluorescence yield of fluorescently labeled amino acids diffusing through the excitation zone. In some embodiments, when a thin adhesive layer is near (e.g., within 10 or 20 or 30 nm of) the edge of distal opening of the reaction well, the thickness and composition of the thin adhesive layer may be selected to provide optimal enhancement of fluorescence excitation in the excitation zone. In some embodiments, the solid state substrate comprises a plurality of thin adhesive layers. For example, the solid state substrate may comprise a first thin adhesive layer between a first opaque metal layer and a second opaque metal layer, and a second thin adhesive layer between the second opaque metal layer and a dielectric layer such as SiO₂ or SiN.

In some embodiments, the solid state substrate comprises a multilayer structure having a plurality of layers for various purposes. Exemplary multilayer structure include a substrate having the following layers from cis side to trans side listed left to right:
(1) SiN (30 nm), Cr (5 nm), Au (300 nm)
(2) SiN (30 nm), Al (200 nm), Cr (5 nm), Au (300 nm)
(3) SiN (30 nm), SiO₂ (200 nm), Cr (5 nm), Au (300 nm)
(4) SiN (30 nm), SiO₂ (200 nm), Al (200 nm), Cr (5 nm), Au (300 nm)
(5) SiN (30 nm), Al (400 nm)

Throughholes may be fabricated in solid state membranes in a variety of materials including but not limited to, silicon nitride, silicon dioxide (SiO₂), and the like. Although silicon nitride is often symbolized as Si₃N₄ (indicating a Si:N stoichiometry of 3:4), silicon/nitrogen mixtures having other stoichiometric ratios of silicon and nitride may be used. For example, a Si:N stoichiometry close to 3:4 but between 3:4 and 4:4 may have lower structural stress than Si₃N₄.

In general, the methods and substrates of the present invention do not comprise or require protein nanopores or lipid bilayers, thereby avoiding their complexity and instability problems.

Solid state throughholes may be prepared in a variety of ways, as exemplified in the references cited above. In some embodiments a helium ion microscope may be used to drill synthetic throughholes in a variety of materials, e.g. as disclosed by Yang et al, Nanotechnology, 22: 285310 (2011). A chip that supports one or more regions of a thin-film material, e.g. silicon nitride, that has been processed to be a free-standing membrane is introduced to the helium ion microscope (HIM) chamber. HIM motor controls are used to bring a free-standing membrane into the path of the ion beam while the microscope is set for low magnification. Beam parameters including focus and stigmation are adjusted at a region that is adjacent to the free-standing membrane, but on the solid substrate. Once the parameters have been properly fixed, the chip position is moved such that the free-standing membrane region is centered on the ion beam scan region and the beam is blanked. The HIM field of view is set to a dimension (in microns) that is sufficient to contain the entire anticipated reaction well pattern and sufficient to be useful in optical readout (i.e. dependent on optical magnification, camera resolution, etc.). Optionally, the ion beam is then rastered once through the entire field of view at a pixel dwell time that results in a total ion dose sufficient to remove all or most of the membrane autofluorescence, if any (e.g., see WO 2014/066905). The field of view is then set to the proper value (smaller than that used above) to perform lithographically-defined milling of either a single throughhole or an array of throughholes that aligned with the corresponding reaction wells. For example, the throughholes may be made to be coaxial, or not coaxial, with the corresponding reaction wells. The pixel dwell time of the pattern is set to result in throughholes of one or more predetermined diameters, which are optionally determined through the use of a calibration sample prior to sample processing. This entire process is repeated for each desired region on a single chip and/or for each chip introduced into the HIM chamber.

The depth and diameter of a reaction well, together with the type(s) and thickness(s) of the one or more opaque metal layers of the substrate, may also be selected to achieve an acceptable level of, or to minimize, excitation light in the well away from the distal opening and towards the proximal throughhole. Generally, the intensity of light incident on the distal opening of a well becomes exponentially weaker as it progresses more deeply in to the well towards the throughhole, so that most of the reaction volume is substantially dark, especially near the proximal throughhole. This is particularly so when an opaque metal layer surrounds the distal opening of the reaction well. A benefit of this phenomenon is that a substantial portion or all of the fluorescently labeled polypeptide strand in the reaction well is protected from unwanted excitation by incident light. This reduces background fluorescence and unwanted modification or degradation of the fluorescently labeled strand. Thus, a deeper well provides greater protection from the incident light, so that a greater proportion of the reaction well is substantially light-free than for a reaction well having the same diameter but smaller depth. A deeper well also provides more space for a longer fluorescently labeled strand to be sequenced. Similarly, a well with a smaller distal opening provides greater darkness in the well away from the distal opening than is provided with a larger distal opening. These general trends are illustrated for example in Table 2 below, which shows light intensities measured in a simulation using Lumerical (March 2020) software available from Lumerical Inc., Vancouver, Canada. More specifically, Intensity was calculated for a well depth 50 nm from the throughhole of the wells (having aluminum side walls) as a fraction of incident light intensity (640 nm) at the distal openings.

TABLE 2

| Well | Well | |
|---|---|---|
| Diameter | Depth | |
| (nm) | (nm) | Intensity |
| 50 | 150 | 4 × 10⁻⁴ |
| 100 | 150 | 1.5 × 10⁻² |
| 50 | 250 | 5 × 10⁻⁸ |
| 100 | 250 | 1 × 10⁻⁴ |

In some embodiments, each reaction well has a combination of depth and minimum diameter exemplified by the combinations in Table 3 below.

TABLE 3

| | Minimum |
|---|---|
| Depth (nm) | Diameter (nm) |
| 150-1000 | 30-150 |
| 150-800 | 30-120 |
| 600-1000 | 30-120 |
| 200-1000 | 50-150 |
| 200-800 | 50-150 |
| 200-1000 | 80-150 |
| 200-800 | 80-150 |
| 400-1000 | 80-150 |
| 200-800 | 80-120 |
| 400-1000 | 80-120 |
| 600-1000 | 80-120 |

The substrate, especially surfaces of the substrate that will be in contact with reaction components may also be coated with one or more coatings to impart desirable properties, such as inertness, non-reactivity, or non-affinity towards buffer or reaction components such as exopeptidase, fluorescently labeled polypeptide strands, released amino acids, and/or other reaction components or buffer components.

In some embodiments, one or more coatings may be applied to the surfaces of the substrate (also referred to herein as “inner surfaces” of the substrate) that may contact one or more buffer components and/or reaction components that are used or are generated in methods of the present invention. For example, such coatings may help passivate surfaces of the reaction wells to reduce their affinity towards exopeptidases and/or amino acids. Such coatings may also be used to protect metal components from oxidation or other degradative processes, or to reduce electroosmotic flow (EOF) of buffer ions along such surfaces that can create aqueous flow along the walls of the well.

In some embodiments, the inner surfaces of the reaction well, the inner surfaces of the throughhole, or the inner surfaces of both the reaction well and throughhole comprise at least one coating. In some embodiments, a single coating is applied. In some embodiments, a plurality of coatings is applied. In some embodiments, when a plurality of coatings is applied, the coatings are the same. In some embodiments, when a plurality of coatings is applied, the coatings are not the same. Such coatings may have any suitable thickness selected by the user. For example, a coating may have a thickness from 1 nm to 20 nm, or from 1 nm and 10 nm, or from 2 nm to 10 nm, or from 5 nm to 10 nm. Preferably, the coating thickness is substantially uniform, as may be provided by a variety of methods, such as atomic layer deposition (ALD).

In some embodiments, a coating comprises an inorganic coating. In some embodiments, an inorganic coating comprises a film comprising HfO₂, Al₂O₃, SiO₂, TiO₂, SiN, or Pt. Such a coating may be made by any suitable method. For example, such a coating may be added by ALD. Such coatings are particularly suitable for coating a variety of metal surfaces, such as aluminum, copper, and gold, and also for coating a variety of other types of material surfaces, such as silicon and silicon nitride. In some embodiments, in which the substrate comprises a gold layer, the gold surface may be coated with an organic thiolate compound. See for example Li et al., Bioconjugate Chem. 24(11):1790-1797 (2013). In some embodiments, in which the substrate comprises a non-gold metal or metal oxide layer, the metal or metal oxide surface may be coated with a phosphonic acid-containing compound such as taught by Mutin et al., Chemical Materials 16:5670-5675 (2004), Gao et al., Langmuir 12:6429-6435 (1996), and Zoulalian et al., J. Physical Chemistry B 110:25603-25605 (2006).

In some embodiments, the substrate comprises a dynamic coating comprising polyvinyl pyrrolidone, which may be present in the buffer in which the sequencing methods of the present invention is performed. Such a coating may be particularly suitable for coating SiN, SiO₂, and metal oxides, for example. In some embodiments, such a coating may reduce non-specific binding of exopeptidase, amino acids, or other buffer or reaction components.

In some embodiments, the solid state substrate comprises a plurality of reaction wells as described in this disclosure, each of which may contain a fluorescently labeled polypeptide strand for sequencing. The plurality of reaction wells may be arranged in any configuration, such as a random or non-random configuration, and are usually disposed in a plane. In some embodiments, the reaction wells are configured as an array to facilitate the performance of a plurality of sequencing reactions in parallel. In some embodiments, the array comprises a plurality of reaction wells arranged in a linear array. In some embodiments, the array comprises a plurality of reaction wells arranged in a 2-dimensional array of rows and columns. In some embodiments, reaction wells are spaced regularly, e.g., in a rectilinear pattern in which parallel rows are perpendicular to parallel columns (i.e., analogous to x and y axes that are 90 degrees apart). In some embodiments, the rows are not perpendicular to the columns. For example, the rows may be parallel to each other, but the columns may extend at a non-90 degree angle, such as 45 or 60 degrees relative to the rows. In some embodiments, the reaction wells may be configured as a hexagonal array in which columns of wells extend from the rows at a 60 degree angle relative to the directions of the rows. In some embodiments, adjacent wells in each row are separated by the same distance from each other. In some embodiments, adjacent wells in each column are separated by the same distance from each other. In some embodiments, the spacing between adjacent wells in each row and column are separated by the same distance from each other. In some embodiments, the spacing between adjacent rows are different from the spacing between adjacent columns.

When the solid state substrate comprises a plurality of reaction wells, each well is preferably separated from all other wells by a distance that permits fluorescent signals to be unambiguously detected from each well, without substantial interference from fluorescent signals from any other wells. Usually, the minimum distance between adjacent wells depends on (1) the longest wavelength of fluorescent light being detected, and (2) the pixel resolution of the signal detector.

Optical resolution of light signals from two adjacent light sources is often considered to be achieved when the light sources are separated by a distance that is at least one half of the wavelength (12) of the detected light, even if the dimensions of light source are smaller than V2 (e.g., when a reaction well has a diameter of 150 nm, 100 nm, or 80 nm). However, greater spacing may be preferred for better resolution to minimize cross-talk interference from light signals from adjacent wells. Thus, for fluorescent signals having a wavelength of 700 nm emanating from adjacent well, a minimum inter-well distance of 350 nm (12) might be sufficient to provide adequate resolution of the two signals. However, a larger inter-well distance would likely improve signal resolution and detection accuracy.

The signal detector may have any suitable pixel resolution that is deemed appropriate by the user. For example, if each pixel of a signal detector has an area of 100 nm×100 nm, and each reaction well has a diameter of 100 nm, then the light signals from each well are usually collected using a plurality of detector pixels for each well (e.g., a 3×3 pixel area, or 4×4 pixel area, or 5×5 pixel area, per well), to capture most or all of the photons emitted from each well. Generally, using a larger number of pixels for signal detection from a well will provide higher a photon yield (i.e., higher signal intensity) of fluorescent signals collected from each well, provided that the pixel area is not too close to the next adjacent well.

In some embodiments, reaction wells are separated by at least 1 micrometer, or by at least 1.3 micrometers, or by at least 1.5 micrometers, or by at least 1.7 micrometers, or by at least 2 micrometers. However, substrates having reaction well separation distances that are larger or smaller than these inter-well separation distances may also be used.

In some embodiments, the plurality of wells comprising an array of at least 10 times 10 reaction wells, or at least 30 times 30 reaction wells, or at least 100 times 100 reaction wells, or at least 500 times 500 reaction wells, or at least 1000 times 1000 reaction wells.

As noted above, reacting a fluorescently labeled polypeptide strand with an exopeptidase in a reaction well releases amino acids, which are fluorescently labeled amino acids or include fluorescently labeled amino acids, from the distal end of the strand. During the exopeptidase reaction with the fluorescently labeled strand, the trans side of the substrate is illuminated with excitation light to create a fluorescence excitation zone adjacent to the distal opening of the reaction well, so that fluorescently labeled amino acids that diffuse through the excitation zone emit fluorescent signals that are detected as a function of time. Stated in a different way, the trans side of the substrate is illuminated with excitation light to create a fluorescence excitation zone adjacent to the distal opening of the reaction well. While the substrate is illuminated, the fluorescently labeled polypeptide strand is reacted with an exopeptidase so that amino acids are released serially from the distal end of the strand and diffuse through the fluorescence excitation zone, so that fluorescently labeled amino acids in the excitation zone emit fluorescent signals.

The fluorescent signals are detected as a function of time, whereby an amino acid sequence or fingerprint is determined from the time order of fluorescent signals detected from the released fluorescently labeled amino acids.

The production and detection of fluorescent signals may be accomplished using any suitable detector. The detector comprises an excitation source that emits light to illuminate one or more sequencing reaction wells at the same time or at different times.

Typically, the excitation light comprises light that is monochromatic, i.e., the light comprises a narrow wavelength range. If the excitation source emits light that is not monochromatic, the light may be passed through one or more filters to block undesired wavelengths from impinging on the reaction wells. Exemplary light sources include lasers (e.g., argon lasers), light emitting diodes, laser diodes, and lamps, such as xenon and mercury lamps. In some embodiments, the detector comprises one or more free space lasers. In some embodiments, the detector comprises one or more fibers coupled lasers.

In some embodiments, the detector comprises a plurality of light sources, such as two or more lasers or light emitting diodes, each having a selected emission wavelength or emission wavelength range suitable for producing excitation light for exciting selected fluorescent labels of fluorescently labeled amino acids that diffuse through the excitation zone of each reaction well.

In some embodiments, the excitation light is circularly polarized. In some embodiments, the excitation light is linearly polarized. In some embodiments, the excitation light is non-polarized. In some embodiments, the excitation light comprises light having a wavelength of 488 nm. In some embodiments, the excitation light comprises light having a wavelength of 532 nm. In some embodiments, the excitation light comprises light having a wavelength of 640 nm. In some embodiments, the excitation light comprises light having a wavelength of 730 nm.

In some embodiments, the excitation light is collimated to illuminate one or more reaction wells, such a plurality of reaction wells that may be configured as an array. In some embodiments, the excitation light is focused, such as in a confocal microscope configuration, which may be used for example for detection of fluorescent signals from a single reaction well.

For embodiments employing multiple fluorescent labels, i.e., when a fluorescently labeled polypeptide strand comprises different amino acids comprising different identifying fluorescent labels, the excitation wavelengths may be tailored to balance the relative intensities of emitted light from the different fluorescent labels. Another way to balance the relative intensities of emitted light from the different fluorescent labels is to select the different fluorescent labels based upon the excitation wavelengths produced by the light source or light sources. For example, the intensity of a fluorescent signal emitted by a fluorescent label may be reduced by exciting the label at a wavelength that is shorter than the label's wavelength of maximum absorption (λmax, abs) corresponding to the label's fluorescence emission wavelength. The different fluorescent labels and light source(s) may also be selected to balance and optimize the resolution of emission signals of the different fluorescent labels. This can be accomplished by choosing labels with emission wavelengths that are as far apart from each other as deemed necessary to distinguish signals from the different labels. For example, in some embodiments, labels are chosen with emission wavelength maxima that are at least 20 nm, or at least 25 nm, or at least 30 nm, greater or less than the nearest emission wavelength maxima of the other labels in the fluorescently labeled amino acids. The choice of labels may also depend on the excitation wavelengths provided by the one or more light sources.

Fluorescence detection may be accomplished using any of a variety of detection modes. Suitable light detectors include, for example, avalanche photodiode detectors; photomultipliers; charge-coupled devices (CCDs), such as intensified CCDs (iCCDs) and electron-multiplying CCDs (emCCDs); complementary metal oxide semiconductor (CMOS) detectors; confocal microscopes; and diode array detectors. Typically, detectors such as CCDs and diode arrays comprise a 2-dimensional array of pixels for collecting fluorescent signals from the reaction wells. As discussed above, the fluorescent signals from a reaction well are usually collected by multiple pixels in the detector, to maximize the collection of photons from that well so that each fluorescent label may be correctly identified. The pixels are usually designed to detect photons over a spectrum of wavelengths that encompass the wavelengths of all of the fluorescent labels that will be released by exopeptidase cleavage of the fluorescently labeled strand.

Fluorescence signals are usually monitored continuously for greatest yield of the detected fluorescent signals. Fluorescence signals are detected and/or recorded using a frame rate that is faster than the duration of fluorescent signals emitted by each released amino acids passing through the excitation zone. The frame rate may be selected by taking into account fluorescent signal strengths and durations of individual photons or photon bursts emitted by the fluorescently labeled amino acids. Fluorescence signals are usually measured after subtraction of background/baseline fluorescence that is measured in the absence of fluorescent amino acids.

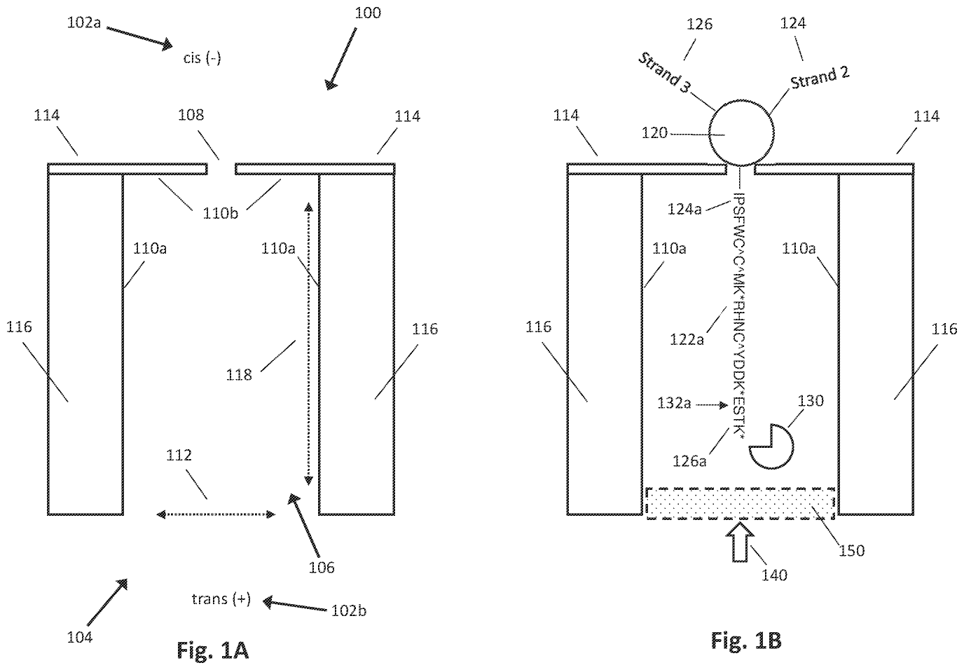

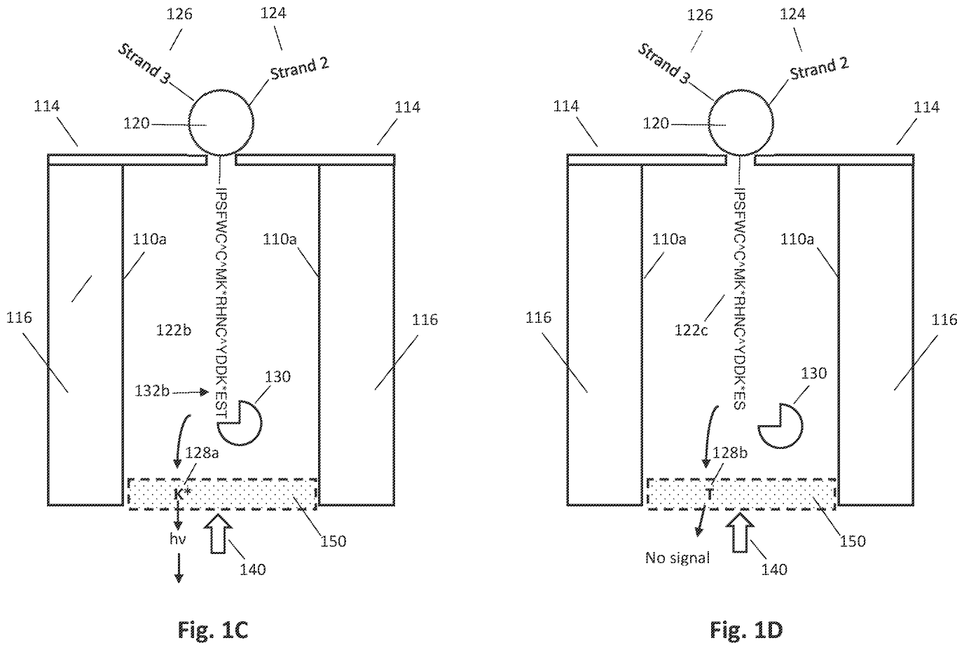

An exemplary solid state substrate comprising a reaction well of the invention is illustrated in the cross-sectional views in FIGS. 1A to 1D. Substrate 100 comprises a cis side 102a and a trans side 102b. Substrate 100 further comprises a reaction well 104 that defines a reaction volume 106. Reaction well 104 comprises (i) a proximal throughhole 108 extending between the cis side and the trans side of the substrate, (ii) one or more side walls 110a and 110b, and (iii) a distal opening 112. Proximal throughhole 108, which may be cylindrical or non-cylindrical, may be provided as an opening passing through a thin membrane layer 114.

Solid state substrate 100 further comprises an opaque metal layer 116 that substantially blocks excitation light from penetrating into the reaction volume and from penetrating from the trans side to the cis side of the substrate. Although FIGS. 1A-1D depict a solid state substrate comprising a single opaque metal layer, the solid state substrate may comprise additional layers and materials, as discussed elsewhere herein.

With reference to FIG. 1A, reaction well 104 is cylindrical, although reaction wells may have other, non-cylindrical shapes. Reaction volume 106 is defined and enclosed by side walls 110a of layer 116, by side walls 110b of thin membrane layer 114, and by the diameter of the reaction well, which is indicated by a dotted horizontal double-headed arrow spanning distal opening 112. The depth of reaction well 104 is illustrated by a dotted vertical double-headed arrow 118.

The minimum diameter of each reaction well should be large enough to allow at least one exopeptidase molecule to diffuse, from the trans side of the substrate, through the distal opening and into the reaction well, and to bind to and digest the distal end of the fluorescently labeled polypeptide strand. Thus, the minimum diameter of each reaction well is or may be made to be at least as large as the smallest dimension or cross-section of the exopeptidase that is used. For example, for an exopeptidase having x-y-z dimensions of 6 nm×6 nm×6 nm, a minimum diameter of 50 nm or 100 nm or 150 nm are each sufficiently large to provide ample space for an exopeptidase to diffuse into a reaction well and serially digest amino acids from the distal end of a fluorescently labeled polypeptide strand.

When an electric field is imposed across the substrate to influence the movement or position of carrier particles loaded with fluorescently labeled polypeptide strands, cis side 102a is associated with an anodic (negatively charged) electrode, and trans side 102b of substrate 100 is associated with a cathodic (positively charged) electrode.

The cis and trans sides of the solid substrate are contacted with one or more aqueous buffers. In some embodiments, the buffers on the cis and trans sides of the substrate may be the same except for the presence of carrier particles (if present) on the cis side and exopeptidase molecules (if present) on the trans side. In some embodiments, at least the cis side buffer includes a protein denaturant or unfolding agent, such as SDS, that extends the fluorescently labeled polypeptide strands into a more linear configuration for exopeptidase access and transit through the throughhole. In other embodiments, the buffers on the cis and trans sides of the substrate are different from each other.

Buffer compositions are provided that are suitable for the sequencing methods of the present invention. Typically, buffers contain buffer molecules, such as HEPES, MOPS, Tris, Na-Acetate and phosphate, for example, to maintain a selected pH (e.g., see Sigma-Aldrich Catalog regarding “Good buffers”). Buffer molecule concentrations of 5 mM to 100 mM are typically useful, although higher or lower concentrations can also be used. Salts and other additives, such as NaCl, LiCl, KCl, and glycerol (e.g., 10 mM KCl to 1 M KCl and/or 1-60 or 1-70 volume percent glycerol) and the like can also be included if desired, as well as appropriate cofactors for the particular enzymes that are used (e.g., MgCl₂ or MnCl₂ for some exopeptidases). In some embodiments, buffer compositions are constituted to maintain the pH substantially constant at a value in the range of 5.0 to 8.8, although buffers with higher or lower pH values may also be used.

To deliver a fluorescently labeled polypeptide strand to a reaction well, an aqueous solution comprising one or more carrier particles, each comprising one or more attached fluorescently labeled polypeptide strands, is contacted with the cis side 102a of substrate 100. Each fluorescently labeled polypeptide strand comprises (i) a proximal end that is attached to the carrier particle, (ii) a distal end that is cleavable by an exopeptidase, and (iii) at least one fluorescently labeled amino acid comprising a fluorescent label. A voltage bias is applied across the substrate using a set of electrodes that establish an electric field from the cis to the trans side of the substrate, typically with one or more anodic electrodes (anodes) on the cis side and one or more cathodic electrodes (cathodes) on the trans side of the substrate. The electric field attracts a carrier particle to the throughhole of a reaction well, so that the distal end of a fluorescently labeled polypeptide strand on the carrier particle is drawn into and through the proximal throughhole into the reaction volume of a reaction well.

However, the carrier particle does not pass through the throughhole. Since the smallest dimensions of the carrier particles are larger than the smallest diameter of a throughhole, or the carrier particles are otherwise too large to pass through the throughhole, the carrier particles remain on the cis side of the substrate. Furthermore, when the throughholes are dimensioned to allow only one fluorescently labeled polypeptide strand to enter each reaction well via a throughhole, or if the carrier particle has a sufficiently low loading density of fluorescently labeled polypeptide strands, only one fluorescently labeled polypeptide strand is present in the reaction well for digestion by an exopeptidase molecule.

The carrier particle and attached fluorescently labeled polypeptide strand may be held (maintained) in place by maintaining a mild voltage bias across the substrate (between the cis and trans sides) to keep the carrier particle adjacent to, or pressed against, the cis side of the throughhole. The carrier particle is not covalently bonded to the throughhole. The voltage bias is sufficiently mild that it does not cause the fluorescently labeled polypeptide strand to be released from the carrier particle. The mild voltage bias may be the same as, or different from, the voltage bias that is used to attract the carrier particle to a throughhole to deliver an attached polypeptide strand to a reaction well.

FIG. 1B shows an exemplary reaction well in which a carrier particle 120 has been moved by an electric field (by a voltage bias) to a location adjacent to the cis side of throughhole 108. Carrier particle 120 comprises three fluorescently labeled polypeptide strands 122a, 122b, and 122c. Each strand is attached to the carrier particle by a proximal end, as illustrated for strand 122a by proximal end 124a, and a distal end that is cleavable by an exopeptidase, as illustrated for strand 122a by distal end 126a. Strand 122a has a contiguous amino acid sequence NH₂-IPSFWC\hat{ }C\hat{ }MK*RHNC\hat{ }YDDK*ESTK* (SEQ ID NO: 1), wherein the N-terminus is attached to carrier particle 120, the C-terminal lysine (K) is the distal end, and “\hat{ }” and “*” denote distinguishable fluorescent labels on cysteine and lysine amino acids, respectively. For FIGS. 1B to 1D, all lysine (K) and cysteine amino acids in strands 122a, 122b, and 122c are fluorescently labeled amino acids each of which comprises a different fluorescent label that distinguishes each kind of amino acid (C and K) from each other.

With continued reference to FIG. 1B, an exopeptidase may be introduced by contacting a second aqueous solution that comprises exopeptidase molecules with trans side 102b of substrate 100 so that an exopeptidase molecule can bind to the distal end of the fluorescently labeled polypeptide strand in a reaction well, cleave amino acids and release them serially from the distal end of the strand. Exopeptidase molecule 130 binds distal end 126a of polypeptide strand 122a and cleaves the peptide linkage (indicated by arrow 132a) between a C-terminal lysine and an immediately adjacent threonine.

During reaction of the exopeptidase with the fluorescently labeled polypeptide strand, the trans side of the substrate is illuminated with excitation light 140 to create a fluorescence excitation zone 150 adjacent to the distal opening of the reaction well. It should be noted that fluorescence excitation zone 150, which is illustrated as a shaded region spanning across the diameter of the distal opening and extending both outside and inside the reaction volume of the reaction well, does not have discrete boundaries. Rather, the intensity of incident light in the excitation zone is most concentrated in the vicinity of the distal opening, approximately as shown in FIG. 1A, and rapidly diminishes at positions further within or outside of (above) the reaction well (for example, see Table 2 and related discussion above). However, the diameter of the excitation zone is the same, or substantially the same, as the diameter of the distal opening of the reaction well. In other words, the excitation zone does not extend to regions of the solid state substrate beyond the diameter of the distal opening. The 3-dimensional intensity profile of incident light for a reaction well will also depend on the composition(s) and other characteristics of the solid state substrate material(s) around the well (e.g., aluminum, gold, or other material). Thus, as used with reference to the present invention, “adjacent to the distal opening of the reaction well” is intended to refer to the space that is both immediately above and immediately below the plane that passes across the distal opening (and defines the distal end of the reaction volume), such as depicted edge-on by arrow 112 in FIG. 1A, and any other nearby illuminated space within the reaction well that causes emission of detectable fluorescent signals by released fluorescently labeled amino acids diffusing through that space.

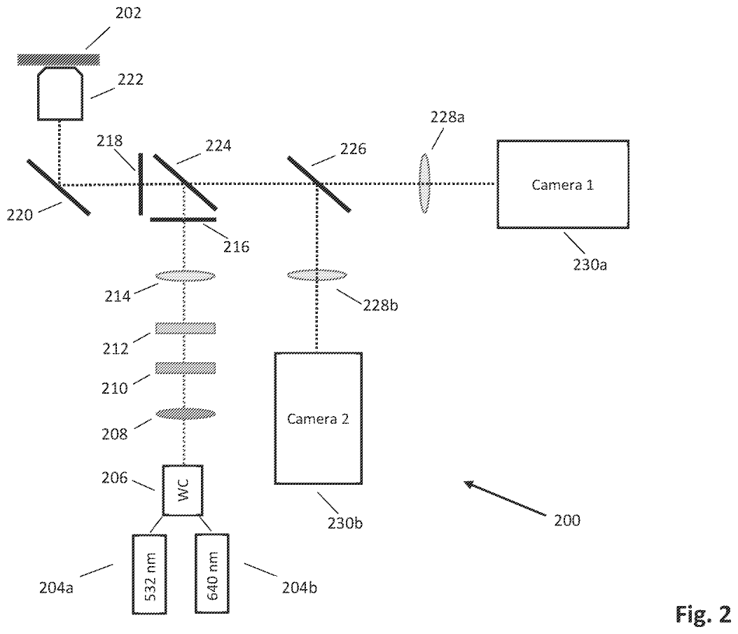

With reference to FIG. 1C, a released amino acid 128a (shown as K*) diffuses out of the reaction volume and through fluorescence excitation zone 150. While in the excitation zone, fluorescently labeled lysine 128a emits fluorescent signals in the form of multiple (a plurality of) photons (hν) that are detected as a lysine by a detector (see FIG. 2, discussed further below). Notably, passive diffusion provides a sufficient mechanism by which released fluorescently labeled amino acids can reach the excitation zone for fluorescence excitation and detection. In the present invention, there is no need for active bulk flow of aqueous solution into or out of the reaction well during exopeptidase cleavage and detection. In some embodiments, active bulk flow of aqueous solution into or out of the reaction well during exopeptidase cleavage is excluded.

Exoproteolytic cleavage and release of the initial C-terminal lysine (K) from strand 122a produces shorter strand 122b having at its distal end a C-terminal threonine (T). Reaction of the distal end of strand 122b with exopeptidase 130 cleaves the peptide linkage (indicated by arrow 132b) between the C-terminal threonine (T) and an immediately adjacent serine (S), producing shorter strand 122c and a released amino acid 128b (shown as T), as shown in FIG. 1D. The released non-labeled amino acid 128b diffuses out of the reaction volume and through fluorescence excitation zone 150, without emitting a fluorescent signal. Exopeptidase 130 is then ready to cleave the next C-terminal amino acid from strand 122c in the same way as for the first two amino acids. After digestion of strand 122a is complete a series of fluorescent signals is obtained corresponding to the ordering of cysteine amino acids (C) and lysine amino acids (K), i.e. KKCKCC, from the C-terminus to the N-terminus of polypeptide 122a, which sequence may be used to identify, or provide a fingerprint, of polypeptide 122a. The identification of polypeptide 122a from fingerprint “KKCKCC” is accomplished by looking up polypeptide 122a in a database which associates polypeptides of interest, e.g. polypeptides derived from human proteins, with fingerprints, or signatures, such as “KKCKCC,” as, for example, taught by Joo et al, U.S. patent publication US2015/0185199.

As digestion continues along the fluorescently labeled polypeptide strand, amino acids are released one-by-one, some of which are fluorescently labeled amino acids, C or K. The released amino acids exit the reaction wells by diffusion at a rate that is much greater than the cleavage rate of the exopeptidase, so that the released fluorescently labeled amino acids serially enter and pass through the excitation zone into bulk solution on the trans side of the reaction well. Unproductive diffusion of amino acids through the proximal throughhole is substantially avoided due to blockage of the proximal throughhole by the carrier particle that is attached to the fluorescently labeled polypeptide strand.

If an exopeptidase molecule dissociates from a fluorescently labeled strand before the fluorescently labeled strand has been completely digested, then another exopeptidase from solution binds to the distal end of the fluorescently labeled strand and continues digestion. Digestion continues until the fluorescently labeled strand is so short that the exopeptidase stops digesting the fluorescently labeled strand or until the cleavage reaction or illumination is otherwise terminated.

Exopeptidase-mediated digestion of the fluorescently labeled polypeptide strands is allowed to proceed for a selected digestion time, or until the yield of reliable fluorescent signals has diminished by a certain amount or below a selected minimum quantity threshold or quality threshold.

After exopeptidase-mediated cleavage (also sometimes referred to as “exopeptidase-mediated digestion” or “exopeptidase digestion”) of the one or more fluorescently labeled polypeptide strands in one or more reaction wells is finished, the reaction wells may be re-loaded with new fluorescently labeled polypeptide strands by applying a reverse voltage bias to the substrate to move the carrier particle away from the throughholes, so that any remaining fluorescently labeled polypeptide strands are removed from the reaction volumes, and then applying a new voltage bias to (across) the substrate to reload each reaction well with a different fluorescently labeled polypeptide strands into each the reaction well (i.e., so that the distal end of a different fluorescently labeled polypeptide strand is delivered into each the reaction well) for reacting with an exopeptidase. The new fluorescently labeled polypeptide strands that are loaded into the reaction wells may be from the same or different carrier particles relative to the carrier particles that provided fluorescently labeled polypeptide strands in the previous exopeptidase cleavage round. In some embodiments turning off the forward voltage bias that holds the nanoparticle at the throughhole is sufficient to remove the nanoparticle. Multiple rounds (cycles) of loading fluorescently labeled polypeptide strands into reaction wells and sequencing the strands using exopeptidase-mediated cleavage may be performed until a desired amount of sequence data has been collected or until the sequencing cycles are no longer sufficiently productive.

In some embodiments, reaction wells are loaded with fluorescently labeled polypeptide strands using a voltage bias of about 250 mV to 500 mV to move the attached carrier particles to the throughholes of the reaction wells, although smaller or larger voltage biases may also be used. In some embodiments, carrier particles are held adjacent to the throughholes using a voltage of about 250 mV to 500 mV, although smaller or larger voltage biases can also be used. In some embodiments, carrier particles are moved away (‘ejected”) from reaction wells using a voltage bias of about −250 mV to −500 mV, although smaller or larger negative voltage biases or no voltage bias may also be used.

One advantage of re-using the carrier particles to deliver multiples fluorescently labeled polypeptide strands to one or more reaction wells is that a large number of fluorescently labeled polypeptide strand can be sequenced from a single solid state substrate and single polypeptide sample. Another advantage is that collecting sequence data from a greater proportion of the total polypeptide sample population can improve the completeness of the sequence data, reduce gaps, and/or increase the collection of redundant sequence data to align sequences and formulate optimal consensus sequences.

An exemplary sequencing apparatus 200 comprising a solid state substrate of the invention is illustrated in FIG. 2. A substrate 202 having a cis side and a trans side is placed adjacent to a microscope objective lens 222, such that the trans side of the substrate faces the lens. Excitation light is delivered to, and fluorescent light signals are received from, the excitation zone of one or more reaction wells in substrate 202. Light paths are illustrated by dotted lines.

The apparatus also comprises excitation light sources 204a and 204b to provide excitation light having selected wavelengths, such as wavelengths of 532 nm and 640 nm, respectively, that are selected to excite fluorescent labels of amino acids that are released from a fluorescently labeled polypeptide strand by exopeptidase activity. Here, the apparatus comprises two fiber-coupled laser light sources 204a and 204b. Excitation light beams having different wavelengths from sources 204a and 204b are passed through wavelength combiner (WC) 206 (a fiber based wavelength combiner), fiber optic connector 208, collimating lens 210 (e.g., an achromatic doublet lens, Thorlabs #AC254-060-A), optional shutter 211, and then through quarter wave plate 212 (e.g., an achromatic quarter wave plate, Thorlabs AQWP05M-600), to convert the linearly polarized beam from the lasers into a circularly polarized beam. The light then passes through focusing lens 214 (e.g., an achromatic doublet lens from Thorlabs AC254-400-A), to focus the beam to the back focal plane of the microscope objective lens, and then through multiband excitation filter 216 (e.g., from Chroma Technology ZET 532/640x). After passing through filter 216, the light is reflected off multiband dichroic beamsplitter 218 (e.g., Chroma Technology ZT 532/640rpc) to mirror 220, through microscope objective 222, and onto the trans side of substrate 201. In some embodiments, microscope objective 222 is an oil immersion microscope objective (e.g., Olympus APON60XOTIRF). In some embodiments, objective 222 is a water immersion microscope objective. In some embodiments, objective 222 is an air microscope objective.

Impingement of the excitation light on the trans side of substrate 200, particularly on the distal opening(s) of one or more reaction wells of substrate 200, creates fluorescence excitation zones (see FIGS. 1B-1D) that are adjacent to the distal openings of the reaction wells.

When fluorescently labeled amino acids are released serially from the distal end of a fluorescently labeled polypeptide strand and diffuse out of the reaction volume through the distal opening of a reaction well, the amino acids diffuse through the excitation zone and emit fluorescent light in response to the excitation light. The emitted fluorescent light is collected and focused by microscope objective 222 and is reflected by mirror 220 through multiband dichroic beamsplitter 218, through multiband emission filter 224 (e.g., Chroma Technology ZET 532/640m) to dichroic beamsplitter 226 (e.g., Chroma Technology T635lpxr). In the apparatus of FIG. 2, the emitted fluorescent light that passes through dichroic beamsplitter 226 is focuses by lens 228a (e.g., Olympus SWTLU-C tube lens) and then onto detector camera 230a for detection; and emitted fluorescent light that is reflected by dichroic beamsplitter 226 is focuses by lens 228b and then onto detector camera 230b for detection (e.g., Hamamatsu C13440-20CU Orca Flash 4.0). Detector cameras 230a and 230b are preferably synchronized to properly detect the time order of fluorescent signals from the released fluorescently labeled amino acids.

For more colors, the two cameras may be replaced with a single camera, and a prism may be used instead of a focusing lens 228a, by which emitted light signals are angularly separated by wavelength onto different regions of the detector field for individual quantification and identification of amino acid-specific fluorescent signals. Alternatively, a third and fourth camera may be included with attendant lenses and beam splitters to capture more than two different fluorescent signals.

Sequencing apparatus for use in the present invention may also comprise a computer and software for collecting and processing fluorescent signal data.

Since the portion of the fluorescently labeled polypeptide strand that is in the reaction volume between the reaction well throughhole and the excitation zone is not substantially illuminated, the non-illuminated fluorescent amino acids in that portion of the fluorescently labeled strand do not emit fluorescent light signals, or if they do, such signals are negligible. Only fluorescent amino acids that are excited while in the excitation zone emit a fluorescent signal that is detected by the detector. Also, if the distal opening is defined by a side wall comprising gold, then excitation intensity of the incident light may be greater than for aluminum, providing an enhancement of fluorescence, in other words, a greater flux of fluorescent photons for detection.

The excitation light that impinges on the distal openings of each reaction well may be oriented so as to be orthogonal to the substrate surface, in other words, parallel to the central axis of each reaction well, e.g., when the reaction wells are cylindrical in shape. Alternatively, the excitation light that impinges on the distal opening of each reaction well may be oriented so as to be non-orthogonal to the substrate surface.

The wavelength of the excitation light is also selected to be compatible with the fluorescent labels of the released amino acids, so that when the excitation light impinges on each fluorescent label, the fluorescent label absorbs the excitation light and then emits photons having a wavelength that is longer (lower energy) than the wavelength of the excitation light. The difference between the wavelength of maximum absorption and the wavelength of maximum emission associated with a fluorescent label is known as the Stokes shift.

Fluorescence signals are detected using a detector frame rate that is faster than the shortest time windows during which fluorescently labeled amino acids emit individual photons or photon bursts while they diffuse through the excitation zone. Usually, released fluorescently labeled amino acids enter the excitation zone within 10, or within 20, or within 50 milliseconds, whereas they are released one-by-one by exopeptidase-mediated cleavage at time intervals between about 100 milliseconds and about 10 seconds. Usually, released fluorescently labeled amino acids enter the excitation zone within 10 milliseconds after being cleaved from a fluorescently labeled strand. Frame rates may also be selected based in part on the size and speed of memory, signal to noise, and fluorescent signal strength.

Exopeptidase cleavage rates and the diffusion times (or diffusion speeds) of released fluorescently labeled amino acids may be adjusted by varying reaction parameters such as pH, viscosity, temperature, and choice of exopeptidase. For example, viscosity may be increased by including a viscous additive such as glycerol, e.g., at a concentration of from 1% to 60% (v:v), or from 1% to 70% (v:v), or from 50% to 70% (v:v), in an aqueous buffer on the trans side of the substrate. In some embodiments, an aqueous buffer on the trans side of the substrate comprises about 50% to about 60% glycerol (v:v), or about 50% to 70% (v:v), or about 50% glycerol (v:v), or about 60% glycerol (v:v), or about 70% glycerol (v:v). The presence of an increased viscosity in and around the detection zone can help reduce amino acid diffusion speeds (and provide longer amino acid dwell times) during fluorescence detection, providing several benefits, such as (1) higher fluorescence signals due to the collection of more emitted photons for each fluorescent amino acid passing through the detection zone, (2) higher signal to noise, (3) the ability to use lower laser power if desired, thereby generating less heat, (4) less cross-contamination (if any) from fluorescent amino acids diffusing from a reaction well towards an adjacent reaction well, (5) and the ability to use place reaction wells more closely together in an array.

Each released fluorescently labeled amino acid may be identified from the characteristics of the measured fluorescent signal, such as (1) the particular emission wavelength or peak shape of fluorescence of the fluorescent label associated with each different kind of amino acid, or subset of amino acids, (2) signal intensity, which may be measured as a sum of multiple photons from the same amino acid during transit through the excitation zone of a reaction well, and (3) the absence of contributions of fluorescence signals from any other released amino acid. For example, fluorescently labeled amino acids that diffuse out of the excitation zone of a first reaction well into the excitation zone of a second reaction zone can be excluded from the fluorescent signals detected for the second well based on the trajectory of movement of the fluorescently labeled amino acids towards the second well. Similarly, fluorescently labeled amino acids that diffuse out of, and then return to, the excitation zone of a first reaction well can be excluded from the fluorescent signals detected for the first well based on the trajectory of movement of the fluorescently labeled amino acid returning towards the first well.

Also, the fluorescently labeled amino acids that are used in the present invention may be selected to be moderately susceptible to photobleaching under the illumination conditions of the substrate, so that fluorescently labeled amino acids that diffuse out of the excitation zone are substantially bleached, and thus rendered non-fluorescent, by the incident excitation light after the amino acid label has been detected in the excitation zone of the first reaction well, before it returns to the same excitation zone or diffuses to another excitation zone. Fluorescence signals from inactive reaction wells can be disregarded by computer software.

As a labeled polypeptide passes through a fluorescence excitation zone time series of fluorescent signals are collected for each different kind of fluorescent label attached to the polypeptide. Thus, for example, if only cysteines and lysines were labeled with distinguishable fluorescent dyes, then two time series of fluorescent signals are collected, which form a fingerprint, or signature, of the polypeptide. In some circumstances, a single time series of signals from one dye may be sufficient to identify a polypeptide; in other circumstances, one or more time series of signals may be required to identify a polypeptide. In still other circumstances, the time series of signals from all of the different fluorescent labels may be required to identify a polypeptide. In still other circumstances, the time series may be insufficient to unambiguously identify a polypeptide, for example, because the time series correspond to only a fragment of a polypeptide, the database of signatures does not contain the signature of the polypeptide analyzed, or the like. Algorithms for identifying polypeptides in databases from measured signatures is well-known in the art and guidance for constructing and using such algorithms may be found in the following references that are incorporated by reference: Joo et al, U.S. patent publication US 2015/0185199; Ginkel et al, Proc. Natl. Acad. Sci., 115(13): 3338-3343 (2018); Yao et al, Phys. Biol., 12: 055003 (2015); Kennedy et al, Nature Nanotechnology, 11: 968-976 (2016); Timp et al, Biophys. J., 102: L37-L39 (2012); Kolmogorov et al, PLoS Comput Biol 13(5): e1005356 (2017); and the like.

The present invention also provides kits that may be useful in performing methods of the invention. Generally, a kit may be any delivery system for delivering materials or reagents for carrying out a method of the invention. In the context of reaction assays, such delivery systems include systems that allow for the storage, transport, or delivery of reaction reagents (e.g., fluorescent labels, enzymes, carrier particles, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second or more containers contain fluorescent labels. In some embodiments, a kit may include one or more of the following: an exopeptidase, buffers, and carrier particles that comprise affinity labels such as avidin, streptavidin, etc.

### Example 1

### Solid State Substrates

Substrate Preparation. A 300 μm thick 100 mm double-side-polished silicon wafer is prepared having a 30 nm layer of SiN deposited by low pressure chemical vapor deposition (LPCVD) on each side of the wafer (e.g., from Virginia Semiconductor, Fredericksburg, VA, or Rogue Valley, OR). Negative e-beam resist is spun on each side and the resist on one side (the “front” side) of the wafer is then exposed in an e-beam lithography (EBL) instrument to pattern reaction wells over the SiN layer on that side. The resist is then developed, and unexposed resist is removed. A 5 nm adhesion layer of chromium or titanium is deposited by e-beam evaporation onto the front side of the wafer, followed by e-beam deposition of a selected thickness of an opaque metal layer (e.g., 200 nm of Au or Al) onto the front side of the wafer. The wafer is then placed in a solution that removes the remaining exposed resist (a “lift off” step) from the front side, leaving reaction wells in the metal film layer that have diameters of 40 to 120 nm and well depths of 100 to 250, or other dimensions according to the preference of the user.

The back (non-patterned) side of the wafer is then patterned via conventional photolithography with a positive tone resist to expose a square window aligned with the front EBL features. These features are etched with reactive ion etch (RIE) through the SiN layer of the back side of the wafer down to the Si layer. The wafer is then mounted in a holder with an O-ring that protects the metal-coated side from KOH solution and is then immersed in KOH solution, which preferentially etches down the (100) plane until the opposite SiN membrane is reached, resulting in a free-standing SiN+metal substrate with reaction wells on the front side of the wafer that are open down to the SiN layer.

Reaction Well-Aligned Throughhole Fabrication. The above-described substrate is then loaded into a focused ion beam (FIB) instrument (e.g., a Zeiss Orion NanoFab in GFIS mode with helium) to create throughholes at the base of each reaction well. Throughholes are milled in free-standing SiN membranes as described previously (Marshall et al., Direct and transmission milling of suspended silicon nitride membranes with a focused helium ion beam. Scanning 34:101-106 (2012)). Briefly, the ion beam is aligned and the substrate to be milled is brought into focus on the tool. Throughholes are milled in the free-standing SiN layer of the substrate by exposing points or rastering over shapes in the substrate for a given dwell time and beam current relative to the thickness of the substrate, to target a desired final size and shape. Other tools capable of fabricating small throughholes include TEMs and other varieties of FIB (for example, gallium or gas field ion source (GFIS) neon). The throughholes may also be lithographically formed by overlaying a second EBL patterning step (as described in the above substrate preparation section) subsequent to creating the larger reaction well. Overlay of the second feature over the first feature yields a pattern consisting of the second feature (reaction well) concentrically aligned with the smaller first feature (throughhole). The resulting pattern may be used directly or, if the second feature is larger than a desired final size, it can be reduced with sub-nanometer precision by atomic layer deposition (ALD) of films such as HfO₂, Al₂O₃, SiO₂, TiO₂, SiN, or Pt.

### Example 2

### Detection of Exoproteolytically Released Amino Acids

Polypeptides are purified from a sample after which their cysteine and lysine amino acids are labeled with distinguishable fluorescent dyes as described in Ginkel et al (cited above) and Joo et al, U.S. patent publication, US2015/0185199. N-terminal amino groups of the labeled polypeptides are then reacted with 2-ethynyl-5-alkynylbenzaldehydes as disclosed in Deng et al (cited above) to give N-terminally alkynyl-derivatized labeled polypeptides, which are then reacted with azide-derivatized carrier particles.

A solid state substrate comprising a cis side and a trans side is prepared using the techniques described above. The solid state substrate comprises a 4×4 square array of reaction wells having radially symmetric side walls with an internal diameter of ˜40 nm, and a well depth of about 315 nm. The side walls of the reaction wells are formed by ion bombardment of a membrane defined within the substrate, the membrane comprising, from the trans side to the cis side, planar layers of (1) aluminum (250 nm), (2) titanium (15 nm), and (3) SiN (30 nm). In addition, the trans and cis sides of the membrane, and the inner side walls of the reaction well, are coated with a 10 nm thick coating of SiO₂ that was added by atomic layer deposition. In this example, the internal diameters of the proximal throughhole, the reaction volume surrounded by the radially symmetrical side wall, and the distal opening of each reaction well are approximately the same.

The reaction wells are separated from each other by a pitch of 1500 nm in the x and y directions. The solid state substrate is assembled in a sequencing cartridge such that the cis and trans sides of the substrate were fluidically accessible. The solid state substrate in the sequencing cartridge is positioned as shown schematically for substrate 202 in FIG. 2, so that the distal openings of the reaction wells on the trans side of the solid state substrate are in the focus of the excitation laser beam that passed through microscope objective 222. The fluidic chamber close to the objective is referred to as trans chamber, and the distal chamber is referred to as the cis chamber. Each chamber is filled with a reaction buffer containing 100 mM Na-Acetate pH 5.2, and then 25 pmole of polypeptide-conjugated carrier particles in the same buffer were added to the cis chamber (final concentration 100 pM nanoparticles with ˜7 polypeptide strands/nanoparticle).

The chambers are electrically connected via two electrodes (Ag/AgCl₂ electrodes). These electrodes are used to apply a cis-to-trans 300 mV voltage bias across the substrate to electrophoretically transport the polypeptide-conjugated nanoparticles to the nanopore reaction wells, so that negatively charged fluorescently labeled polypeptide strands attached to the carrier particles are electrophoretically drawn through the proximal throughholes and into the reaction volumes of the reaction wells.

Plugging of the proximal throughholes with carrier particles is monitored by measuring the cis-to-trans current from a starting (open, unplugged) current of approximately 1000 nAmp to a current below about 200 nAmp when the rate of change of the current plateaues. Plugging of all 16 wells is complete in a few seconds. Because the diameters of the carrier particles are greater than the diameters of the reaction wells, the carrier particles are prevented from passing through the reaction wells from the cis side of the substrate to the trans side. In addition, because the carrier particles are sparsely loaded with fluorescently labeled polypeptide strands, no more than one fluorescently labeled polypeptide strand is loaded into each reaction well.

After the proximal throughholes of the reaction wells are plugged, and fluorescently labeled polypeptide strands are loaded into the reaction wells, the trans chamber buffer is replaced with the following exopeptidase reaction buffer: 0.1 M sodium acetate pH 5.2, 50% (w/v) Sucrose, and carboxypeptidase Y (ThermoScientific).

Immediately after introducing the exopeptidase reaction buffer, laser illumination of the trans side of the substrate and camera recording of fluorescence emissions from the distal ends of the reaction wells are started. Camera frame rates for emission signal detection is usually between 200 and 500 frames per second (fps), and laser light intensities at 535 nm and 648 nm are usually from 5 mWatt to 15 mWatt.

This disclosure is not intended to be limited to the scope of the particular forms set forth, but is intended to cover alternatives, modifications, and equivalents of the variations described herein. Further, the scope of the disclosure fully encompasses other variations that may become apparent to those skilled in the art in view of this disclosure.

### Definitions

“Sequence determination”, “sequencing”, “determining amino acid sequence”, “determining a polypeptide sequence”, and similar terms, when referring to polypeptides, includes the determination of partial or full amino acid sequence information of one or more polypeptides. These terms also include determining sequences of subsets of the full set of twenty natural amino acids, such as, for example, a sequence of only cysteine amino acids (C) and lysine amino acids (K) of target polypeptides, or polypeptide sample. These terms also include the determination of the identities, order, and locations of one, two, three or more of the 20 types of amino acids within target polypeptides. In some embodiments, the terms include the determination of the identities, order, and locations of cysteine amino acids (Cs) and lysine amino acids (Ks) within target polypeptides. In some embodiments, the terms include the determination of the identities and order of cysteine amino acids (Cs) and lysine amino acids (Ks) within target polypeptides. In some embodiments, these terms may also include subsequences of target polypeptides that serve as a fingerprints or signatures for the target polypeptides; that is, subsequences that uniquely identify a target polypeptide, or a class of target polypeptides, within a set of polypeptides.

“Target polypeptides” mean one or more polypeptides from a sample whose sequences are to be determined.

“Target sequence” means a sequence of a target polypeptide.

1. A method for determining an amino acid sequence of a polypeptide comprising: providing
(a) a solid state substrate comprising a cis side and a trans side, the substrate comprising a reaction well that defines a reaction volume and comprises
(i) a proximal throughhole extending between the cis side and the trans side of the substrate,
(ii) one or more side walls, and
(iii) a distal opening,
wherein the solid state substrate further comprises an opaque metal layer that substantially blocks excitation light from penetrating into the reaction volume and from penetrating to the cis side of the substrate, and
(b) a carrier particle comprising a fluorescently labeled polypeptide strand that is attached to the carrier particle, wherein the fluorescently labeled polypeptide strand comprises
(i) a proximal end that is attached to the carrier particle,
(ii) a distal end that is cleavable by an exopeptidase, and
(iii) at least one fluorescently labeled amino acid comprising a fluorescent label,
wherein the carrier particle is located on the cis side of the substrate, but does not pass through the throughhole, such that the fluorescently labeled polypeptide strand protrudes through the throughhole so that the distal end of the fluorescently labeled polypeptide strand is in the reaction volume;
reacting the fluorescently labeled polypeptide strand with an exopeptidase so that amino acids are released serially from the distal end of the strand and diffuse out of the reaction volume through the distal opening;
during said reacting, illuminating the trans side of the substrate with excitation light to create a fluorescence excitation zone adjacent to the distal opening of the reaction well, so that fluorescently labeled amino acids in the excitation zone emit fluorescent signals; and
detecting the fluorescent signals as a function of time;
whereby an amino acid sequence is determined from the time order of fluorescent signals detected from the released fluorescently labeled amino acids.

2. The method of any one of the preceding claims, wherein the distal opening of the reaction well has a minimum diameter of at least 30 nm.

3. The method of any one of the preceding claims, wherein the distal opening of the reaction well has a minimum diameter of 50 to 150 nm.

4. The method of any one of the preceding claims, wherein the one or more walls of the reaction well are not tapered.

5. The method of any one of the preceding claims, wherein the one or more walls of the reaction well are substantially cylindrical.

6. The method of any one of the preceding claims, wherein the opaque metal layer comprises gold or aluminum.

7. The method of any one of the preceding claims, wherein the opaque metal layer has a thickness of 100 nm to 600 nm.

8. The method of any one of the preceding claims, wherein the reaction well has a well depth of at least 200 nm.

9. The method of any one of the preceding claims, wherein the reaction well has a well depth of 200 nm to 1000 nm.

10. The method of any one of the preceding claims, wherein the fluorescently labeled polypeptide strand in the reaction volume comprises a fluorescently labeled polypeptide segment containing at least 60 contiguous amino acids.

11. The method of claim 1 wherein the throughhole has a minimum diameter of at least 2 nm.

12. The method of claim 1 wherein the throughhole has a minimum diameter of 2 nm to 50 nm.

13. The method of any one of the preceding claims, wherein the substrate comprises a thin membrane layer that contains the proximal throughhole and has a thickness of between 20 nm and 50 nm.

14. The method of claim 13, wherein the thin membrane layer comprises silicon nitride.

15. The method of any one of the preceding claims, wherein the excitation light has a wavelength of 380 nm or greater.

16. The method of any one of the preceding claims, wherein the solid substrate comprises surface portion(s) that define the reaction volume, and the surface portion(s) comprise at least one surface passivation coating.

17. The method of any one of the preceding claims, wherein one or more side walls of the reaction well comprises at least one of a silicon oxide coating and an aluminum oxide coating.

18. The method of any one of the preceding claims, wherein the fluorescently labeled polypeptide strand comprises at least two different kinds of amino acid, each kind labeled with a distinguishing fluorescent label.

19. The method of any one of the preceding claims, wherein during said reacting, the carrier particle is maintained next to the proximal throughhole by a voltage bias.

20. The method of claim 19, wherein after said reacting, the voltage bias is stopped to allow the carrier particle to move away from the proximal throughhole, so that the remaining fluorescently labeled polypeptide strand is removed from the reaction volume, and then a voltage bias is applied to move the same or a different carrier particle toward the proximal throughhole so that a new fluorescently labeled polypeptide strand is delivered into the reaction well for reacting with an exopeptidase.

21. The method of any one of the preceding claims, wherein the carrier particle is not magnetic.

22. The method of any one of the preceding claims, wherein the carrier particle is magnetic.

23. The method of any one of the preceding claims, wherein the carrier particle comprises a plurality of fluorescently labeled polypeptide strands.

24. The method of any one of the preceding claims, wherein the carrier particle comprises a plurality of fluorescently labeled polypeptide strands having polypeptide sequences that are different from each other.

25. The method of any one of the preceding claims, wherein the solid state substrate comprises a plurality of reaction wells.

26. The method of claim 25, wherein the plurality of reaction wells are configured as a one-dimensional or two-dimensional array.

27. The method of claim 25 or 26, wherein two or more of the plurality of reaction wells each contain a fluorescently labeled polypeptide strand to be sequenced.

28. A method for determining an amino acid sequence of a polypeptide comprising:
translocating through a nanopore a labeled polypeptide wherein at least two different kinds of amino acids are labeled with distinguishable fluorescent labels, and wherein the nanopore comprises a passage through an insulative layer and an opaque layer, the passage through the opaque layer having a diameter through the opaque layer;
illuminating the passage from the direction of the opaque layer with a light beam having a wavelength greater than the diameter of the passage through the opaque layer, so that an excitation zone of non-propagating light is created within the passage through the opaque layer;
digesting the labeled polypeptide in the passage outside of the excitation zone to release amino acids one at a time at a rate less than the expected time of diffusion of the released amino acids out of the passage;
measuring time series of fluorescent signals comprising signals from released labeled amino acids by detecting the signal generated by fluorescent labels as the released labeled amino acids diffuse out of the passage through the excitation zone.

29. The method of claim 28 further including a step of identifying a polypeptide from said time series of fluorescent signals by looking up the polypeptide in a database that associates time series of fluorescent signals with amino acid sequences of known polypeptides.

30. The method of claim 28 wherein said step of digesting includes digesting said polypeptide with an exopeptidase.

31. The method of claim 28 wherein said passage comprises a zero-mode waveguide.

32. The method of claim 28 wherein said step of translocating includes attaching said polypeptide to a carrier particle that guides said polypeptide into said passage and that has a size that prevents its entry into said passage.

33. The method of claim 28 wherein said passage comprises in series from a cis side to a trans side of said insulative and opaque layers a throughhole and a reaction volume, wherein the reaction volume comprises a zero-mode wave guide.
